(19) United States
(12) Patent Application Publication
(10) Pub. No.: US 2008/0292714 A1
(43) Pub. Date: Nov. 27, 2008

(54) BONE TARGETING OF DEGRADABLE DRUG FILLED NANOPARTICLES

(75) Inventors: Joseph R. Garlich, Westfield, IN (US); Donald L. Durden, Decatur, GA (US); Lisa Brannon-Peppas, Austin, TX (US); Tim C. Smith, Indianapolis, IN (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Semafore Pharmaceuticals Inc., Indianapolis, IN (US)

(21) Appl. No.: 12/181,963

(22) Filed: Jul. 29, 2008

Related U.S. Application Data
(63) Continuation of application No. 10/817,728, filed on Apr. 2, 2004, now abandoned.
(60) Provisional application No. 60/460,355, filed on Apr. 3, 2003.

(51) Int. Cl. A61K 9/51; A61K 31/428; A61P 39/00

(52) U.S. Cl. 424/501; 514/367

(57) ABSTRACT

This invention provides a method of modifying a cellular response in a mammal. The method comprises administering to the mammal an effective amount of biodegradable nanoparticles, each of said nanoparticles comprising an active agent, a biodegradable polymer, and a bone targeting agent administering to a mammal an effective amount of a composition comprising a compound absorbed in a biodegradable nanoparticle which is attached to a bone targeting agent. The invention also provides a method for modifying a cellular response in a mammalian cell comprising contacting the mammalian cell with biodegradable nanoparticles. The invention further provides a method of delivering an exogenous substance to a mammal. The method comprises administering to the mammal a composition comprising the exogenous substance absorbed into a biodegradable nanoparticle, wherein the biodegradable nanoparticle is covalently attached to a bone targeting agent. The invention also provides a composition and a process for preparing the composition comprising a biologically active or therapeutic agent of compound, a biodegradable nanoparticle, and a bone targeting agent.

### CROSS-REFERENCE TO RELATED PATENT APPLICATIONS

This is a continuation of co-pending U.S. patent application Ser. No. 10/817,728, filed Apr. 2, 2004 claiming the benefit of U.S. Provisional Patent Application No. 60/460,355, filed Apr. 3, 2003. The disclosure of the '728 application and the '355 application are incorporated by reference.

### STATEMENT REGARDING FEDERALLY SPONSORED RESEARCH AND DEVELOPMENT

This invention was made in part with Government support under Grant Number IR43CA101545-01 awarded by the National Cancer Institute. The Government may have certain rights in this invention.

### FIELD OF THE INVENTION

This invention pertains to compositions and methods for the targeted and controlled delivery of active agents to mammalian cells, for example, bone and bone marrow cells employing nanoparticles.

### BACKGROUND OF THE INVENTION

Targeted delivery of active agents, e.g., therapeutic substances, to a specific location of the body is a goal that has met with only limited success. One of the goals in the treatment of disease is to specifically deliver the therapeutic agent exclusively to the area requiring treatment. Extensive effort has been put forth in rational drug design to produce a compound that will selectively treat the specific ailment, but the biological variety that exists within a living organism means the drug usually does not interact exclusively with the tissue requiring treatment. The classical example of this challenge is the treatment of cancer with chemotherapeutic agents. Chemotherapy usually focuses on killing the active cancer cells, typified by uncontrolled growth, at a faster rate than it kills the healthy cells that may coincidentally be growing at the same time.

Another goal in the treatment of disease is the controlled release of the therapeutic substance over an extended period of time, in order to provide a sustained treatment using a single dose rather than multiple doses. Several products on the market are available that use biodegradable products to release a drug into the body over a specific period of time. For example, parenteral depot systems, such as Lupron Depot, Nutropin Depot, and Trelstar Depot listed in the FDA Orange Book, are products that provide a controlled release from poly(lactic-co-glycolic) acid (PLGA) microparticles of a therapeutic agent over a period of one month or more. However, the injection of this formulation is localized, typically a parenteral injection, and the drug is released into the blood stream and distributed throughout the body rather than exclusively to the area of requiring treatment.

Substantial work has been done on microparticles, and the commercial Depot products are typically of a size on the order of microns. Smaller nanoparticles have only recently been described and result from the realization that sub-micron particles could find utility in particular drug applications (Jain, Biomaterials, 21, 2475 (2000)).

The combination of targeted delivery and controlled release of a therapeutic agent at a specific location of the body has only recently been explored in a viable manner by the use of biodegradable nanoparticles targeted to a specific tissues in the body. For example, Cheresh et al. (Science, 296, 2404 (2002)) have demonstrated the delivery of therapeutic genes to cancer cells using a lipid-based nanoparticle with an integrin antagonist as the targeting moiety. The particles delivered genes selectively to angiogenic blood vessels in mice, although the rate of release of the gene therapy was not addressed. Edwards et al. (International Patent Publication WO 03/088950) describes targeting nanoparticles that contain a therapeutic, diagnostic, or prophylactic agent by attaching a ligand that binds to cell surface receptors. Examples of ligands include hormones, antibodies, or ligands for specific cell surface receptors such as lutenizing-hormone-releasing-hormone (LHRH). Specifically, the hormone LHRH helped direct the delivery of toxins to the targeted cells in order to kill the cells. Russell-Jones et al. (International Patent Publication WO 00/66090) describes the targeted delivery of nanoparticles containing toxins or cytotoxic agents using folic acid or an analogue thereof that coats the surface of the nanoparticle. Unger et al. (International Patent Publication WO 03/087389) describes the delivery of antisense polynucleic acids contained within a nanoparticle and directed by a “cell recognition” component that targets the nanoparticles to receptors on cellular structures. Esenaliev et al. (U.S. Pat. No. 6,165,440) describes the targeted delivery of nanoparticles with antibodies to antigens in the tumor vasculature. The nanoparticles are metal, carbon, graphite, polymers or liquid loaded with an absorbing dye, or porous gas-filled particles. The nanoparticles are delivered to the solid tumor blood vessels and irradiated with a laser or non-laser source (e.g., ultrasound) in order to perforate the blood vessels and allow a chemotherapeutic agent that was added separately to more easily enter the solid tumor.

Stella et al. (Proceed. Int'l. Symp. Control. Rel. Bioact. Mater., 28, 6306 (2001)) and Russell-Jones et al. (Proceed. Int'l Symp. Control. Rel. Bioact. Mater., 28, 7109 (2001)) have reported that vitamin B12 and folate provide a four-fold increase in the targeting of HPMA (N-2-hydroxypropylmethacrylamide) nanoparticles to hybridoma cells in mice. Shin et al. (Proceed. Int'l Symp. Control. Rel. Bioact. Mater., 28, 5058 (2001)) have shown biotin-conjugated particles of biotinylated pullulan acetate can preferentially target hepatic carcinoma cells over fibroblast cells. The permeability of tumors to particle uptake appears to be dependent not only on the specific targeting agents, but also on the particle surface charge. An analysis of liposome uptake has shown that the adenocarcinoma tumors and melanoma tumors studied preferentially took up cationic liposomes over anionic and neutral liposomes (Proceed. Int'l Symp. Control. Rel Bioact. Mater., 27, 428 (2000)).

The targeted delivery of biodegradable nanoparticles to the bone and bone marrow of a living organism has not been realized. The only example of bone targeting nanoparticles are thin-coated iron oxide particles for magnetic resonance imaging (Drug Development Research, 54, 173 (2001)). In this example, the particle size was very small, on the order of 10 nanometers, and did not provide for the controlled release of a therapeutic agent. The iron-containing nanoparticles were exposed to bisphosphonates in aqueous solution, and physiochemical surface adsorption was postulated. Some limited localization was found in male Wistar rats. Using radiolabeled iron-59, the animal studies (18 hours after injection) indicated at best 6% of injected dose in the skeletal system with almost half going to the spleen and liver. The attachment of the bisphosphonate to the nanoparticle was not optimized or characterized. Roberts and Kozlowski (U.S. Pat. No. 6,436,386) describe a PEG molecule covalently attached to both a bisphosphonate and a drug candidate for targeting bone tissue. The product was not described to form nanoparticles nor was the compound described as a component for forming nanoparticles.

The foregoing shows that there exists a need for particles, especially nanoparticles, for the targeted delivery and controlled release of therapeutic agents to the bone and bone marrow of an animal or human. The present invention provide such particles. The advantages of the invention, as well as additional inventive features, will be apparent from the description of the invention provided herein.

### BRIEF SUMMARY OF THE INVENTION

The invention provides a method of modifying a cellular response in a mammal comprising administering to the mammal an effective amount of biodegradable nanoparticles, each of said nanoparticles comprising an active agent, a biodegradable polymer, and a bone targeting agent. The invention also provides a method for modifying a cellular response in a mammalian cell comprising contacting the mammalian cell with biodegradable nanoparticles.

In addition, the invention provides a method of delivering an exogenous substance to a mammal The method comprises administering to the mammal an effective amount of biodegradable nanoparticles comprising the exogenous substance, a biodegradable polymer, and a bone targeting agent.

The invention provides a composition comprising a active agent, a biodegradable nanoparticle, and a bone targeting agent. The invention further provides for a process for preparing a biodegradable nanoparticle comprising a active agent, a biodegradable polymer, and a bone targeting agent.

### BRIEF DESCRIPTION OF THE DRAWINGS

FIG. 1 depicts the chemical structure of EDTMP.

FIG. 2 depicts the chemical structure of DOTMP.

FIG. 3 depicts the chemical structure of ABDTMP.

FIG. 4 depicts the chemical structure of BAD.

FIG. 5 depicts the chemical structure of MTX-BP.

FIG. 6 depicts the chemical structure of CF-BP.

FIG. 7 depicts chemical structures of bone targeting agents A-N, according to an embodiment of the invention.

FIG. 8 depicts a method for modifying a phosphonate in a bone targeting agent to introduce an amine binding site.

FIG. 9 depicts an alkylation reaction to modify a phosphonate in a bone targeting agent.

FIG. 10 depicts a modification of EDTMP and DOTMP.

FIG. 11 depicts the chemical structure of pifithrin-α.

FIG. 12 depicts the chemical structure of pifithrin-β.

FIG. 13 depicts chemical modification of a PLGA polyester to form two possible activated esters, bearing R* groups.

FIG. 14 depicts a reaction scheme for preparing pifithrin-α.

FIG. 15 depicts a reaction scheme for preparing a PEG aminodiphosphonic acid, V.

FIG. 16 depicts a reaction scheme for preparing PEG-Fluorescein complex, Z.

FIG. 17 depicts reaction schemes for preparing amine phosphonic acid bone targeting agents AD and AG.

FIG. 18A is a scanning electron micrograph (SEM) of hydroxyapatite particles as received from BioRad at a magnification factor of 700×. FIG. 18B is an SEM of hydroxyapatite particles as received from BioRad at a magnification factor of 30,000×

FIG. 19A is an SEM of hydroxyapatite particles after treatment with nanoparticles that do not contain bone targeting agents at a magnification factor of 700×. FIG. 19B is an SEM of hydroxyapatite particles after treatment with nanoparticles that do not contain bone targeting agents at a magnification factor of 30,000×.

FIG. 20A is an SEM of hydroxyapatite particles after treatment with bone targeting nanoparticles, in accordance with an embodiment of the invention, at a magnification factor of 700×. FIG. 20B is an SEM of hydroxyapatite particles after treatment with bone targeting nanoparticles, in accordance with an embodiment of the invention, at a magnification factor of 30,000×.

FIG. 21A is an SEM of hydroxyapatite particles after treatment with bone targeting nanoparticles, in accordance with an embodiment of the invention, at a magnification factor of 700×. FIG. 21B is an SEM of hydroxyapatite particles after treatment with bone targeting nanoparticles, in accordance with an embodiment of the invention, at a magnification factor of 30,000×.

FIGS. 22A and 22B are graphs illustrating the release profiles of nanoparticles containing doxirubicin and epirubicin, respectively.

FIG. 23 depicts reaction scheme for preparing a PEG-modified polyester, AJ, or a phosphonic acid modified polyester, AI.

FIG. 24 depicts a scheme for preparing an aminodiphosphonic acid-PEG-PLGA, AM, for the use in nanoparticle preparation.

FIG. 25 depicts a reaction scheme for preparing an aminotetraphosphonic acid-PEG-PLGA, AP, for the use in nanoparticle preparation.

### DETAILED DESCRIPTION OF THE INVENTION

The invention provides biodegradable nanoparticles that are sufficiently modified with anionic calcium binding moieties in order to target or deliver the nanoparticles to a selected tissue, cell, or organ, e.g., the bone, of an animal. Contained within the nanoparticles are one or more active agent, e.g., therapeutic agents, that can modify a cellular response in the bone or bone marrow of a patient. Such delivery of active agents to the bone can be used as a sensitizer to enhance the effects of chemotherapy or radiation treatment of bone and marrow diseases, or to deliver chemotherapeutic agents to the bone and bone marrow. Alternatively, the therapeutic agent can be a chemoprotectant to prevent bone marrow suppression during chemotherapy or radiation treatment of nonbone/nonmarrow diseases, or can deliver agents that encourage bone growth or regrowth. By targeting the biodegradable nanoparticles to the bone, the therapeutic agent's effect can be localized and the delivery of the drug at this site controlled over a specific period of time.

One aspect of the invention is the controlled delivery of a drug at one or more selected rates over an extended period of time. The delivery can be via oral, transdermal, or parenteral (injectable or implantable) routes. These controlled release systems release enough drug to maintain the drug level in the body at an effective therapeutic concentration over a long period of time. The advantages of such release systems in general are the avoidance of toxic or ineffective drug levels, the most efficient use of the drug itself, and fewer drug doses than with systems of conventional administration. The drug can be released at a constant period over time, at a pulsatile rate of delivery, or at different rates, e.g., an initial delivery of a bolus of drug followed by a slower controlled rate of delivery.

In an embodiment of the invention, a therapeutic agent is delivered by a biodegradable nanoparticle that contains the therapeutic agent. By “biodegradable” is meant a compound that can be decomposed, degraded, or otherwise destroyed by biological or biochemical processes. The products of these biodegradable polymers may be completely broken down and removed from the body by normal metabolic pathways. Biodegradable polymers have advantages over other carrier systems in that they need not be surgically removed when drug delivery is completed and that they can provide direct drug delivery to the systemic circulation. The active agent and polymer may be combined in a number of different ways depending upon the application of interest. Particulate formulations have the widest applicability to the widest variety of formulation needs, including oral delivery, intramuscular injection, subcutaneous injection, intravenous injection, and site-specific delivery, such as to the surface of a bone during surgery.

This invention utilizes any suitable biodegradable polymer, such as biodegradable polymers that are currently in use or are being developed for controlled drug delivery in vivo. For example, the biodegradable polymer can be a polyester, a polylactone, a polycarbonate, a polyamide, or a polyol, preferably a polyester. The polyester can be composed of poly(lactic acid), commonly known as PLA, poly(glycolic acid), commonly known as PGA, and their copolymers, commonly known as poly(lactic-co-glycolic) acid or PLGA. The nanoparticles composed of PLGA can have any suitable ratio of PLA and PGA, e.g. a lactic acid:glycolic acid ratio (e.g., molar ratio) of about 95:5 to about 5:95, preferably of about 75:25 to about 25:75, or more preferably of about 50:50. The PLGA copolymer can be a random copolymer or block copolymer of lactic acid and glycolic acid, The block copolymers can have 2, 3, 4, or more blocks of PLA and PGA. The lactic acid component can be racemic, enantomerically enriched with the D or the L isomer, or enantiopure. The hydrolysis of these polyesters, by both nonenzymatic and enzymatic esterase-based pathways, leads to glycolic and lactic acids which are easily metabolized in the citric acid cycle. While degradation by nonenzymatic processes has no dependency on the chirality of the polyesters, the rate of cleavage by biological esterase shows some dependency on the chirality of the lactic acid. Therefore, the enantiomeric ratio of the polyester could effect the degradation rate of the nanoparticle and by extension the release rate of the therapeutic agent, and provides for flexibility in controlling the rate of drug delivery. The ends of the polymer chain may also be end-capped with any group known in the art, such as, for example, methyl or lauryl esters.

By varying the monomer ratios in the polymer processing and by varying the processing conditions, the resulting polymer can exhibit drug release capabilities for months or even years. Increasing the ratio of PLA increases the relative hydrophobicity of the nanoparticle, while increasing the ratio of PGA increases the hydrophilicity. The resultant nanoparticle can therefore bind active agents with a wide range of hydrophobicities and hydrophilicities, and the subsequent release of the active agents can be optimized by controlling the monomer ratios and processing conditions. In addition, crystallinity, molecular weight, and amounts of any residual solvents used in the preparation may also affect the release rates of active agents.

In another embodiment, the biodegradable nanoparticle comprises poly(ethylene glycol) or poly(ethylene oxide), commonly known as PEG or PEO, which is a polyether formed either from ethylene glycol or ethylene oxide as a monomer. The molecular weight of the PEO or PEG can be any suitable weight, e.g., it can range from as low as 400 to as high as 5,000,000. Preferably, the molecular weight is from about 700 to about 100,000, more preferably from about 1000 to about 20,000, and even more preferably from about 3000 to about 5000. PEG is currently being used in drug delivery for suppositories, prostaglandin formulations, and contraceptive sponges, and as a wound healing laminate. Although PEG is not degraded in the body, it has been shown to be safe for biological applications, with no detectable toxic or cumulative effects of intravenous injection of PEG even after repeated doses ranging up to 90 mg/kg per day. Many PEGs can be prepared with a functional group that provides for attachment of another moiety. These activated PEGs have been designed for attachment to lysine amino groups, making them ideal for use with proteins, peptides, and enzymes. Protein-PEG conjugates are more stable to proteolyses and denaturation than the native proteins. Modified PEGs provide increased thermal stability and aqueous solubility, e.g., when modified with immunoglobulin G.

In the invention, PEG can be incorporated into the nanoparticle by any suitable approach, e.g., as a block copolymer of the biodegradable polymer graft or as an attachment (e.g., covalent) to the nanoparticle or its surface, as a blend of PEG and the biodegradable polymer used during formation of the nanoparticle, or as a coating of the PEG onto the nanoparticle surface. The PEG can be associated with the polymer by ionic, covalent, coordinate, hydrogen bonding, van der Waals, and other intermolecular forces, or be a simple blend. When PLGA, PLA, or PGA and PEG are utilized, e.g., as a triblock copolymer, the PEG or PEO often, though not necessarily, is the central block, and the polyester chains are at either end of the polymer. Studies have evaluated the effect of the length of a central PEG block (J. Contrl. Rel., 24, 81 (1993)) as well as the length of outer PLA blocks (Macromolecules, 29, 50, 57 (1996)) on water absorption and degradation of these copolymers. Kissel et al. have explored the synthesis of these tri-block materials, in vitro degradation, drug delivery, in vitro biocompatibility, and in vivo biocompatibility, as well as the microenvironment of PLA-PEO-PLA microparticles during degradation. The biocompatibility studies have shown that PLA-PEO-PLA polymers show very similar and minimal adverse tissue reactions. Drug delivery studies which compared in vitro delivery of bovine serum albumin from microparticles prepared from PLA-PEO-PLA and PLGA-PEO-PLGA polymers showed that the PLGA-containing polymers exhibited fairly continuous release profiles while PLA-containing polymers had two phases of release more typical of simple PLGA microparticles (J. Contrl. Rel., 32, 121 (1994)). Release studies of cytochrome C and FITC-dextran from PLGA-PEO-PLGA microparticles also showed continuous release in vitro (J. Contrl. Rel., 39, 315 (1996)).

The use of PEG in nanoparticle preparations advantageously provides for a degree of “stealthiness” that nanoparticles without PEG do not have. The nanoparticles having a PEG component avoid detection and sequestration by the mononuclear phagocyte system and the reticuloendothelial system and subsequent elimination in the liver or kidneys. Accordingly, the stealthiness increases the residence time and the effectiveness of the nanoparticles in drug treatment. The use of PEG or PEO on proteins or nanoparticles has been shown to increase the circulating lifetime of these foreign species. Examination of the biodistribution of nanoparticles containing PEG shows greatly enhanced circulation times for PLGA-PEG nanoparticles over PLGA nanoparticles alone (Li et al., J. Cntrl. Rel., 39, 315 (1996)). Specific cellular uptake studies have shown that a surface layer of PEG will avoid premature capture of nanoparticles by the mononuclear phagocyte system with PLGA-PEG copolymers, with PEG segments of 5,000 molecular weight showing the greatest protection (Jaeghere et al., J. Drug Target., 8, 143 (2000)). The types of active agents recently successfully incorporated and released from PLGA-PEG copolymers include rhodamine B (Panoyan et al., Proc. Intl. Sym. Cntrl. Rel. Bioact. Mat., 28, 5120 (2001)), taxol (for microparticle preparation) (Das et al., J. Biomed. Mat. Res., 55, 96 (2001)), adriamycin (Lie et al. J. App. Poly. Sci., 80 1976 (2001)), doxorubicin (Yoo et al., J. Contrl. Rel., 70, 63 (2001)), and VEGF (for microsphere preparation) (King et al., J. Biomed. Mat. Res., 51, 383 (2000)).

Unmodified PLA nanoparticles that are injected intravenously are taken up by cells of the mononuclear phagocyte system, mainly the Kuppfer cells (Fawaz et al., Pharmaceutical Research, 10, 750 (1993)). This may naturally concentrate these particles close to liver parenchymal cells and facilitate biliary clearance and enterohepatic circulation. In general, nanoparticles without surface modification are rapidly cleared from the blood and are concentrated in the liver, spleen, and bone marrow. Unmodified nanospheres of PLGA (75:25 lactic acid:glycolic acid) can be prepared especially for site-specific delivery based on their size (Scholes, et al., J. Cntrl. Rel., 23, 145 (1993)). Biodistribution of injected colloidal carriers is highly dependent upon their size and their surface properties. For example, for targeted administration to the lung, particles should be several microns in diameter. Modification of the surfaces of colloidal particles with PEG will modify the uptake of particles and reduce immediate liver sequestration.

By “nanoparticle” is meant a particle of approximately spherical shape measuring less than about 1000 nm in diameter. Several aspects of the invention rely on the size of the nanoparticle, including the rate of decomposition, the rate of release of the therapeutic agent, the ability to access the bone tissue of a mammal, and the ability to avoid macrophages in the circulatory system. Furthermore, smaller nanoparticles are known to cross into the cellular matrix, typically by endocytosis, and the size requirements of the nanoparticles is an important characteristic in transportability. Nanoparticles may enter a cell via the cellular caveloae, typically 20-60 nm openings that participate in receptor-mediated uptake processes, and via receptor-mediated endocytosis in clathrin-coated pits, typically in the range of 150-200 nm (see Unger et al., supra.) Furthermore a lining of cells in the bone functions as a marrow-blood barrier to limit the accessibility of exogenous large substances to the bone (Talmage, Am. J. Anat., 129, 467-76 (1970)). Consequently, an important aspect of the invention is the size and size distribution of the nanoparticles. In one embodiment, the nanoparticles of the invention have a diameter of about 10 nm to about 1000 nm. In a preferred embodiment, the nanoparticles have a diameter of about 50 to about 500 nm, more preferably from about 100 to about 400 nm, and even more preferably from about 100 to about 250 nm. The size distribution of the nanoparticles is also important since different sizes produce different release rates and different drug loading levels. The size range of the nanoparticles can be narrow, broad, or multimodal. The number of nanoparticles within a given size range can be greater than about 75%, greater than about 85%, greater than about 95%, or greater than about 99%. For example, if greater than 99% of the nanoparticles were within the range of 150-250 nm, then the distribution might be considered narrow, whereas greater than 75% of the nanoparticles within the range of 10-1000 nm might be considered broad. Alternatively, the size distribution of particles can be characterized by the relative polydispersity. Relative polydispersity is a value determined by the Coulter Nanosizer described below, and indicates the relative distribution around the median diameter. A relative polydispersity of 1 indicates a monodisperse sample, while increasing values indicate a broader distribution within the sample. The relative polydispersity can be less than about 5, preferably less than about 3, and more preferably less than about 2.

In another aspect of the invention, the composition comprises a bone targeting agent. By “bone targeting agent” is meant a chemical structure or ligand that has a high affinity for calcium ions in hydroxyapatite, the major constituent of bone. The composition of the invention can be targeted, in an embodiment, to calcium deposits in regions of the body other than bone, such as calcium deposits in the arteries, heart, kidney, or gall bladder. However, the bone targeting agent ideally selectively binds to bone tissue. A bone targeting agent of the invention is attracted to the bone tissue of the subject, preferably binds to the bone with a higher affinity than non-bone tissues, and remains bound for a certain length of time thereby delivering the composition to a bone environment. In other words, the bone targeting agent preferably binds to bone tissue with at least 2-fold greater affinity (e.g., at least 3-fold, at least 5-fold, at least 10-fold, or at least 25-fold greater affinity) than to a non-bone tissue. The bone targeting agent preferably reversibly binds to bone tissue, meaning that the bone targeting agent is eventually released from bone and expelled from the body.

The bone targeting agent preferably remains bound to bone tissue for a sufficient period of time to allow the attached nanoparticle to deliver the therapeutic agent(s) to the target cells (e.g., bone marrow cells). The bone targeting agent can remain bound to bone for about 1 or more days (e.g., about 2 days, about 3 days, or about 7 days) to about 1 year or more (e.g., about 330 days, about 365 days, or about 400 days), after which the bone targeting agent is expelled from the body. The bone targeting agent can remain bound to bone for about 7 or more days (e.g., about 7 days, about 14 days, or about 21 days) to about 6 months or more (e.g., about 90 days, about 120 days, or about 150 days). For example, a bone targeted nanoparticle can remain bound to the bone for 30 days, during which time the drug is released and the nanoparticle degrades. After about 45 days the bone targeting agent would be released from the bone and eventually excreted, e.g. after 30 or 45 days of treatment Thus, a bone targeting agent for use in the invention can be selected based on binding kinetics to bone tissue. Candidate bone targeting agents can be screened in vitro by determining affinity to bone tissue (e.g., hydroxyapatite) in, for example, a multi-well format. Candidate bone targeting agents also can be screened in vivo by assessing the rate and timing of excretion of candidate bone targeting agents from the body. In this respect, the bone targeting agent preferably is expelled from the body via the kidneys.

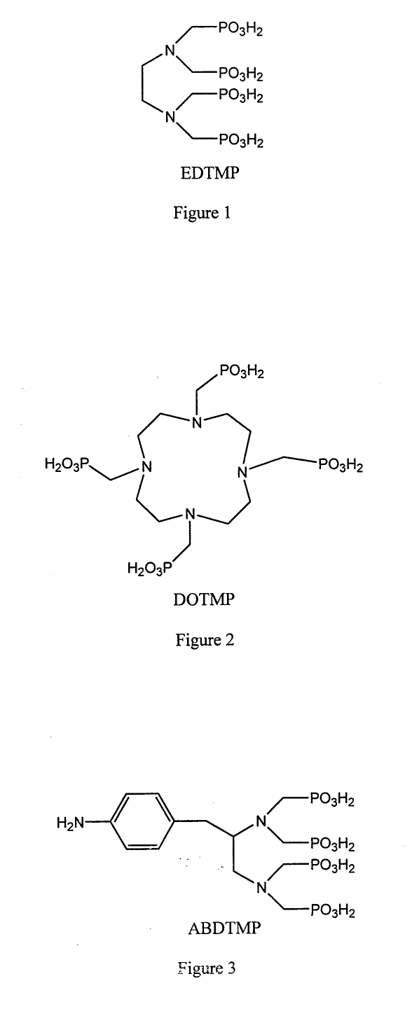

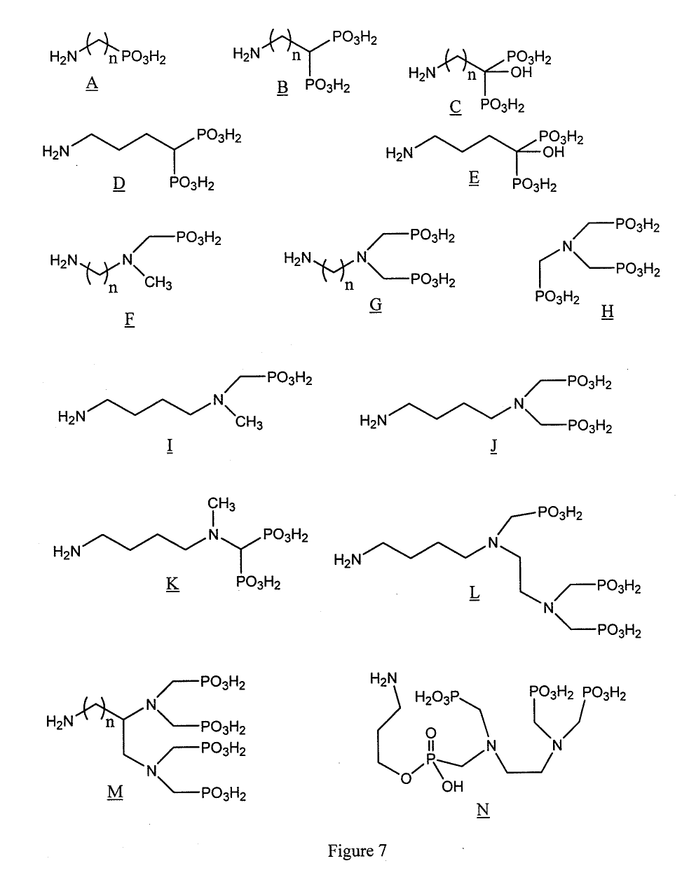

The bone targeting agent desirably is selected from the group consisting of a phosphate, a phosphonate, a bisphosphonate, a hydroxybisphosphonate, an aminomethylenephosphonic acid, an acidic peptide, or a combination thereof. The bone targeting agent of the invention can carry one, two, three, or more of these groups. For example, the bone targeting agent can be a phosphonate, meaning that the bone targeting agent may comprise one phosphonate, two phosphonates, or three or more phosphonates. One suitable bone targeting agent for use in the invention is EDTMP (ethylene diamine-N,N,N′,N′-tetrakis(methylenephosphonic acid), the chemical structure of which is set forth in FIG. 1), currently FDA approved (Quadramet™) as the radioactive ¹⁵³Sm complex for delivering a selective radiation dose to bone metastases for pain palliation. EDTMP is a phosphonate that contains four phosphonic acid groups, and is therefore a tetraphosphonate. Compounds such as ¹⁵³Sm-EDTMP are selectively localized in bone where tumors are present versus normal bone in a ratio of more than 10:1, probably because metabolic turnover of calcium is very high in the metastatic region. The ¹⁵³Sm-EDTMP reportedly is rapidly taken up by the skeleton in osteoblastic bone metastases and cleared from the plasma. That portion of the compound that does not accumulate in the skeleton reportedly is rapidly excreted, and excretion is almost complete within 6 hours after administration (Jimonet et al., Heterocycles, 36, 2745 (1993)). The pain palliation is thought to be due to the radiation originating from the isotope bound to the osteoblastic bone metastases having some effect on the nearby metastatic tumor cells. Another clinically useful bone targeting system is DOTMP (the chemical structure of which is set forth in FIG. 2), now in Phase III clinical trials (termed STR, skeletal targeted radiation) as the radioactive ¹⁶⁶Ho complex designed to deliver large doses of radiation selectively to the bone marrow for the treatment of multiple myeloma. It should be noted that the radioactive ¹⁶⁶Ho-DOTMP complex localizes in the skeletal system and irradiates the nearby bone marrow which houses the malignant myeloma cells. Like the ¹⁵³Sm-EDTMP system, the phosphonate that does not localize in the bone is cleared through the urine and out the body. See FIG. 7 of Bayouth et al., J. Nucl. Med., 36, 730 (1995).

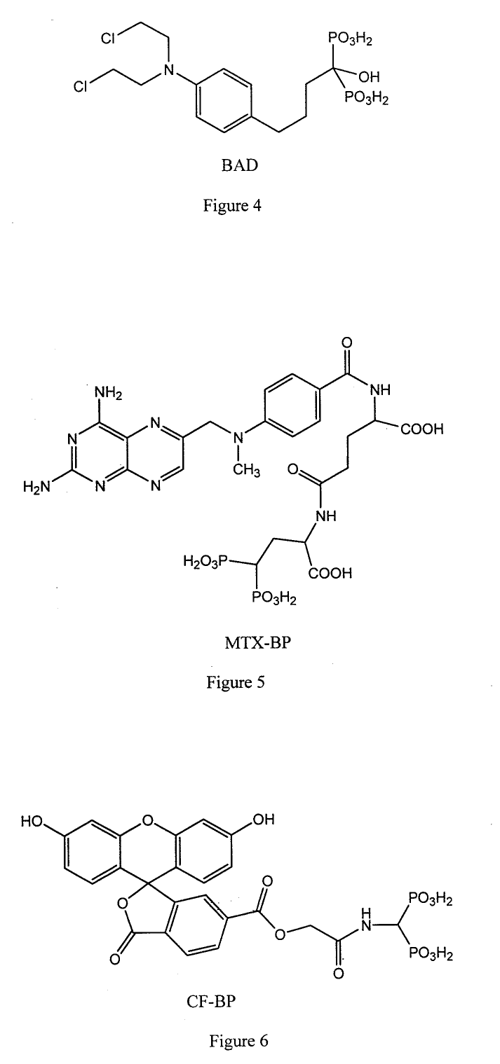

Preferably, the bone targeting agent is a polyphosphonic acid. Polyphosphonic acid has been demonstrated to successfully target biologically-active molecules to bone tissue. For example, conjugation (via isothiocyanato chemistry) of polyaminophosphonic acids, such as ABDTMP (the chemical structure of which is set forth in FIG. 3), to growth factors (to stimulate bone formation) successfully resulted in the targeting of the growth factors to the bones of rats (see, for example, International Patent Publication WO 94/00145). Similarly, bone targeting agents have been coupled to proteins. For example bisphosphonates that were conjugated to human serum albumin successfully delivered the protein to bone in vitro (Biotechnol. Prog., 16, 258 (2000)) and in vivo (Biotechnol. Prog., 16, 1116 (2000)). The utility of bone targeting agents extends beyond delivery of proteins to bone and includes, for instance, small therapeutic molecules. A conjugate comprising a bone targeting bisphosphonate and an alkylating agent, such as BAD (the chemical structure of which is set forth in FIG. 4), has been generated (see, for example, Wingen et al., J. Cancer Res. Clin. Oncol., 111, 209 (1986)). In this molecule, the alkylating agent is not specific in its interaction with its target (DNA), and, thus, there is no requirement for cleavage between the bisphosphonate (i.e., bone targeting agent) and the alkylating moiety. The bisphosphonate-alkylating agent demonstrated efficacy in a rat osteosarcoma model using BAD. Another series of studies have been performed using the antifolate antineoplastic agent methotrexate that has been covalently attached to bisphosphonates, designated MTX-BP and shown in FIG. 5 (see, for example, Sturtz et al., Eur. J. Med. Chem., 27, 825 (1992); Sturtz et al., Eur. J. Med. Chem., 28, 899 (1993); and Hosain et al., J. Nucl. Med., 37, 105 (1996)). Using Tc-99m labeled MTX-BP, it was determined that around 15% of the injected dose was localized in the skeleton after 4 hours with about 61% of the dose being excreted (Hosain, supra). MTX-BP further demonstrated five times greater anticancer activity compared with methotrexate alone in animal models of transplanted osteosarcoma (Sturtz 1992, supra). Similar work has been described using the conjugate CF-BP, a carboxyfluorescein group with an appended bisphosphonate whose chemical structure is set forth in FIG. 6 (Fujisaki et al., Journal of Drug Targeting, 4, 117 (1994)). In this molecule, the CF group is a fluorescent marker to quantitate pharmacokinetics and biodistribution, and is connected to the bone targeting agent through an ester bond which is susceptible to hydrolysis in vivo. Studies in rats injected intravenously indicated that CF-BP localized in the bone and served as a slow release mechanism for CF generated via general hydrolysis of the ester linkage (Fujisaki, supra).

In another embodiment, the bone targeting agent can be a peptide, such as (ASP)₆ and (Glu)₆. The acid-rich peptide sequence of the glycoprotein osteonectin, which is found in abundance in bone and dentin, has a strong affinity to hydroxyapatite (Fujisawa et al., Biochimica et Biophysica Acta, 53, 1292 (1996)). Thus, peptide ligands comprising acidic amino acids are suitable candidates for bone targeting agents. Indeed, (Glu)₁₀, when attached to biotin, successfully recruited labeled strepavidin to hydroxyapatite (described further in Chu and Orgel, Bioconjugate Chem., 8, 103 (1997), and International Patent Publication WO 98/35703). In addition, the biological half-life of the fluorescein isothiocyanate conjugated to (Asp)₆ was 14 days in the femur (Kasugai et al., Journal of Bone and Mineral Research, 15(5), 936 (2000)), which is an acceptable half-life for the bone targeting agent of the invention. Likewise, delivery of estradiol-(Asp)₆ conjugates to bone has been demonstrated in ovariectomized animals with concomitant inhibition of osteoporectic-type bone loss (Kasugai et al., Journal of Bone and Mineral Research (Suppl 1), 14, S534 (1999)). It is believed that the (Asp)₆ tether to bone is metabolized during the bone resorption process mediated by osteoclasts. Therefore, the acidic peptide ligand provides not only a means of recruiting compounds to bone, but also provides a mechanism of slowly releasing compounds to bone cells and surrounding tissue.

Other examples of bone targeting agents include, but are not limited to amino- and hydroxy-alkyl phosphonic and diphosphonic acids; hydroxybisphosphonic acids including alendronate, pamidronate, 4-aminobutylphosphonic acid, 1-hydroxyethane-1,1-diphosphonic acid, and aminomethylenebisphosphonic acid; phosphates such as phytic acid; and aminomethylenephosphonic acids such as N,N-bis(methylphosphono)-4-amino-benzoic acid and nitrilotri(methylphosphonic acid). Nonlimiting examples of some bone targeting agents are shown in FIG. 7.

Preferably, the bone targeting agent is an aminomethylenephosphonic acid. By “aminomethylenephosphonic acid” is meant a compound that contains an —NCH₂PO₃H moiety, where the amino group has one, two, or three methylenephosphonic acid groups attached, and may be further substituted with other chemical moieties. An aminomethylenephosphonic acid may include one or more phosphonic acid groups and one or more amino groups. Examples of these aminomethylenephosphonic acids include but are not limited to the compounds F through N set forth in FIG. 7.

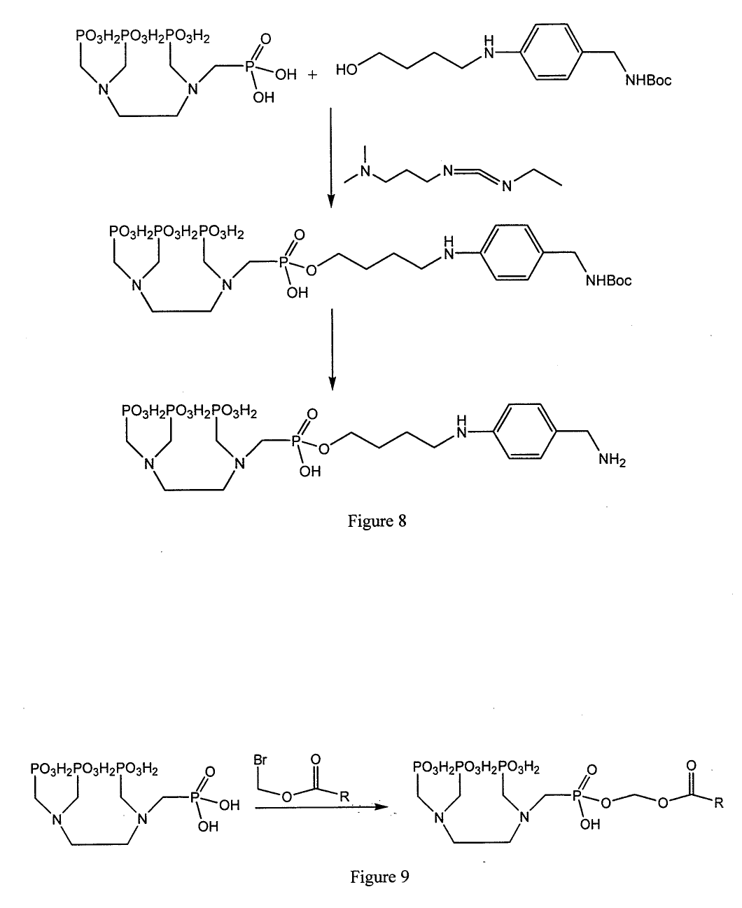

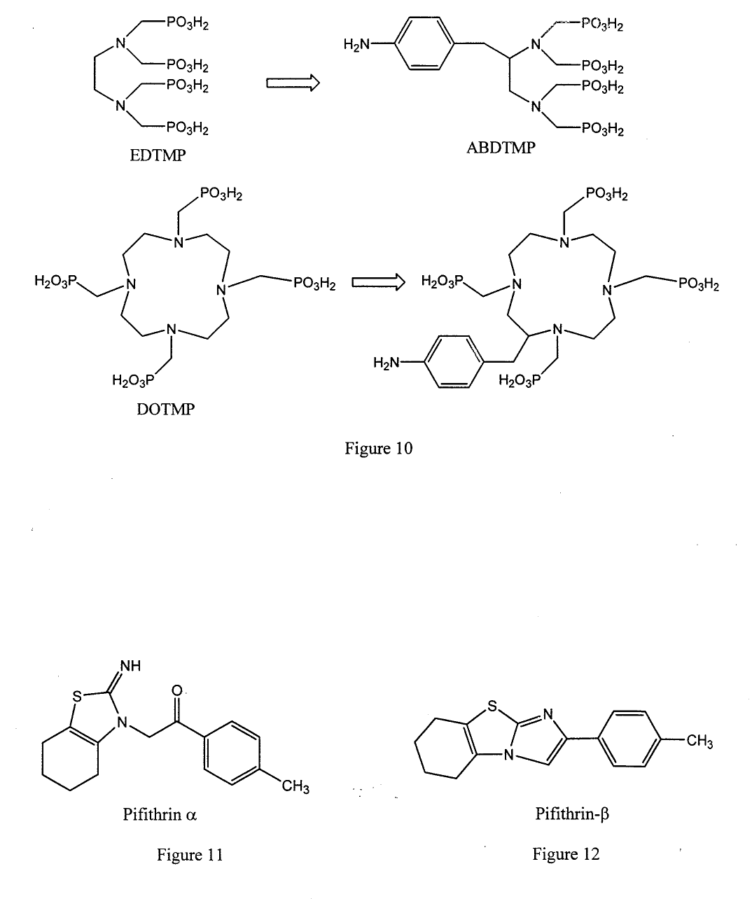

It is envisioned that these bone targeting agents and other bone targeting agents can be attached through one of the heteroatoms or by chemical modification that installs an additional attachment point. For example, EDTMP can be connected to a linker by one of the phosphorous oxygens to create a phosphonate linkage, as illustrated in FIG. 8 (see for example Vieira de Almedia et al., Tetrahedron, 55, 12997-13010 (1999).) The phosphorous oxygen can also be alkylated as shown in FIG. 9, where the R group can have, for example, a pendant amino group, to provide a secondary attachment point for ligation to, for example, an activated PEG. Other types of alkylation that could be utilized in the invention include but are not limited to examples similar to that involving DOTMP, as has been further described in Chavez et al., Biomedical Imaging: Reporters, Dyes, & Instumentation, Contag & Sevick-Muracia, Eds., Proc. SPIE, Vol. 3600, 99-106 (July, 1999), or as shown for other phosphonic acids further described in, for example, U.S. Pat. No. 5,177,064, U.S. Pat. No. 5,955,453, de Lombaert et al., J. Med. Chem., 37, 498-511 (1994), and Iyer et al., Tetrahedron Letters, 30(51), 7141-7144 (1989). Alternatively, for chemical modification, EDTMP can be, for example, modified to generate ABDTMP by installation of an aniline group (as further described in, for example, FIG. 5 of International Patent Publication WO 94/00145). The aniline amine is then available to form, for example, an amide bond. DOMTP could be similarly modified, as outlined in FIG. 10.

The terms “phosphonate, phosphate, and aminomethylenephosphonate” are meant to encompass the phosphonic acids, the phosphoric acids, and aminomethylenephosphonic acids, respectively, as well as any salts, hydrolyzable esters, and prodrugs of the phosphorous-based acids thereof. At the biological pH of 7.4 in the blood, or the more acidic pH around the bone, a certain portion of the phosphate or phosphonate of the bone targeting agent may be deprotonated and replaced with a counterion. Furthermore, the exchange of proton for calcium is an inherent event for the binding of the bone targeting agent to the hydroxyapatite in the invention. However, preparation and administration of the composition containing the bone targeting agent may or may not require complete protonation of the phosphorous acids therein. Therefore, the phosphonic acid, phosphoric acid, and aminomethylenephosphonic acid are drawn and utilized interchangeably with phosphate, phosphonate, and aminomethylenephosphonate. Biologically hydrolyzable esters of the phosphorus-based acids may also be utilized in the in vivo use of the bone targeting nanoparticles. Similarly, prodrugs of the phosphorous-based acids may also be utilized in vivo to mask the acidity of the composition during, for example, formulation and administration.

The nanoparticles can be prepared in any suitable manner. For example, the preparation methods for biodegradable microparticles known in the art can be used to prepare the nanoparticles of the invention. Most preparations are based on solvent evaporation or extraction techniques (see, for example, D. H. Lewis “Controlled Release of Bioactive Agents from Lactide/Glycolide Polymers” in Biodegradable Polymers as Drug Delivery Systems, Marcel Dekker, p. 1 (1990)). The simplest methods involve dissolving the polymer in an appropriate organic solvent and suspending this solution in an aqueous continuous phase which contains an appropriate surfactant. Continuous stirring then allows for evaporation of the organic solvent and hardening of the microparticles. The key factors that control the size and size distribution of these particles are the polymer concentration in the solvent, the amount and type of surfactant, and the stirring rate. This solvent evaporation method is most appropriate for incorporating drugs that are soluble in the same organic solvent as the polyester. In this case, the drug and polymer are dissolved together in the organic solvent and a molecular mixture of polymer and drug will exist in the resulting microparticles (see, for example, Brannon-Peppas, Int'l J. Pharmaceutics, 116, 1, (1995) and Matsumoto et al., J. Cntrl. Rel., 48, 19 (1997)). The solvents used in these techniques include dichloromethane, acetone, methanol, ethyl acetate, acetonitrile, chloroform, and carbon tetrachloride.

Variations on this basic solvent evaporation technique include: (i) solvent extraction, (ii) double emulsions, (iii) oil-in-oil systems, (iv) phase separation or coacervation and (v) multiple emulsion potentiometric dispersion. These variations are used for more water-soluble drugs such as peptides and proteins or to modify the typical release profile seen from biodegradable microparticles.

While microparticles have been prepared using PLA and PLGA for many years, nanoparticles of these materials are fairly new and are the result of modifications of existing preparation techniques. Optimization of new techniques to prepare nanoparticles of PLA and PLGA has been described (Brannon-Peppas et al., J. Nanoparticle Res., 2, 173 (2000)) and production scale-up of such nanoparticles from 100 mg per batch to 100 g per batch has been demonstrated. A preparation of nanoparticles comprising PLGA and PEG has been described (Li et al., J. Cntrl. Rel., 68, 41 (2000)).

The biodegradable nanoparticles can be attached to a bone targeting agent in any suitable manner, such as via a covalent bond between the bone targeting agent and a polyester end group or via a covalent bond to the PEG. In one preferred embodiment, the bone targeting agent is covalently bound to at least about 10% of the PEG, to at least about 25% of the PEG, or at least about 50% of the PEG of the nanoparticles.

The bone targeting agents may be attached to the PEG by any suitable technique known in the art. In one embodiment, the bone targeting agent is attached to PEG by reacting the bone targeting agent with an activated PEG. An activated PEG is a PEG that contains a reactive functionality that may be, for example, displaced or otherwise modified. Formula VI is one example of such an activated PEG

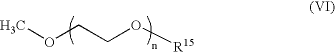

wherein n is an integer from 2 to 2000, preferably from 10-1000, and more preferably from 30-200, and R¹⁵ a organic radical that contains an electrophilically activated leaving group. By “electrophilically activated leaving group” is meant a group that will be attacked by an incoming nucleophile, e.g., an amine or a alcohol, thereby forming a new covalent bond. Examples of R¹⁵ include but are not limited to epoxy groups, aldehydes, isocyanates, isothiocyanates, succinates, carbonates, propionates, butanoates, etc., such as succinimidyl glutarate, succinimidyl, succinimidyl succinamide, succinimidyl carbonate, N-hydroxysuccinimidyl carbonate, propionaldehyde, succinimidyl propionate, succinimidyl butanoate, and the like. In a preferred embodiment, the R¹⁵ is a succinimidyl propionate or succinimidyl butanoate. An additional organic linkage may or may not be present between the activated PEG and the bone targeting agent, as demonstrated in the examples.

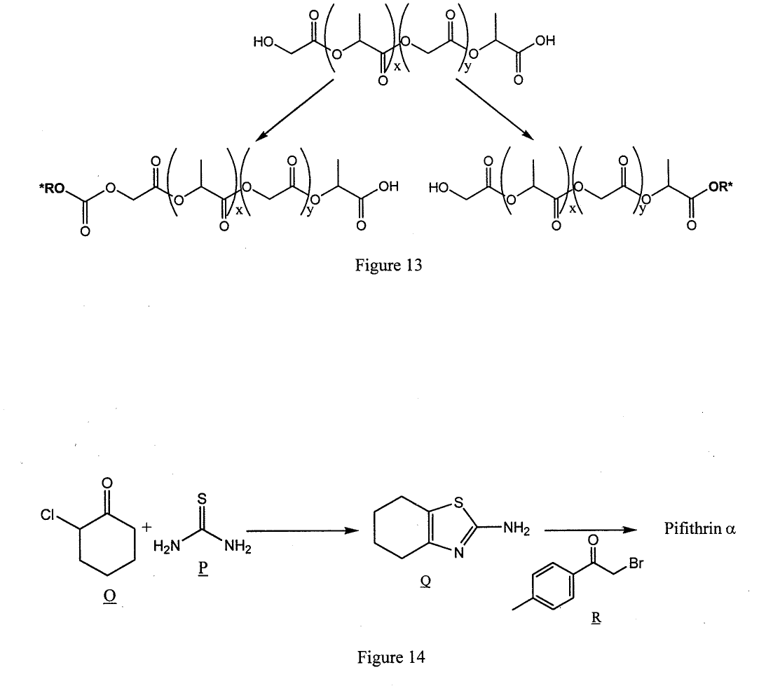

The bone targeting agents may be attached to the polyester by any suitable technique known in the art. In one embodiment, the bone targeting agent is attached to a polyester by reacting the bone targeting group with a polyester containing an activated ester end group, as are known in the art. (See, for example, Yoo, et al., Pharmaceutical Research, 16, 1114 (1999)). The activated ester end group may be present at either end of the polyester, for example, as shown in FIG. 13 (the OR* being a displaceable group.)

In one embodiment, biodegradable nanoparticles comprising an active agent, a biodegradable polymer, and a bone targeting agent can be prepared by a process comprising providing an organic phase, e.g., a suspension or a solution, with one or more of a biodegradable polymer, a PEG, an activated PEG, a bone targeting agent, a PEG-modified biodegradable polymer, a bone targeting agent-biodegradable polymer conjugate, a bone targeting agent-PEG conjugate, a bone targeting agent-PEG-modified biodegradable polymer conjugate, and an active agent therein, with the requirement that the organic phase contains at least one PEG, at least one biodegradable polymer, and at least one active agent, mixing the organic phase. The organic phase is mixed with an aqueous phase, e.g., a suspension or a solution, comprising water and a surface active agent. The organic solvent(s) are removed from the mixture while stirring, thereby recovering the resultant nanoparticles and optionally treating the nanoparticles with a bone targeting agent. The organic solvent or solvents can be any solvent used in the art, preferably a solvent selected from the group consisting of C₁-C₄ alcohols, C₂-C₆ esters, C₂-C₆ ethers, and C₁-C₆ organic acids. The surface active agent in the aqueous layer is any agent used in the art that aids in the formation of the nanoparticles, preferably bovine serum albumin, human serum albumin, or polyvinyl alcohol. The surface active agent may be in any concentration that provides for control of nanoparticle sizes. In one preferred embodiment the bovine serum albumin or human serum albumin is present in the aqueous phase at a concentration of about 5-15 mg/ml. Alternatively, the polyvinyl alcohol can be present in the aqueous phase at a concentration of about 0.5 to 2.0% by volume.

The bone targeting nanoparticles of the invention, in an embodiment, may be prepared by combining a biologically active agent with various combinations of compounds selected from the group comprising a PEG; an activated PEG, for example as shown in Formula VI; a bone targeting agent; a polyester (e.g., PLGA); a bone targeting agent-PEG conjugate, wherein the bone targeting agent is connected to the PEG; a bone targeting agent-polyester conjugate, wherein the bone targeting agent is connected to the polyester; a PEG-modified polyester (e.g. a block copolymer of PEG-PLA-PEG); and a bone targeting agent-PEG-modified polyester conjugate, with the requirement that the resulting nanoparticle contain at least one PEG, at least one bone targeting agent, at least one polyester, and at least one active agent. The nanoparticles may comprise one type of bone targeting agent, or multiple types of bone targeting agents. The use of different bone targeting agents in a nanoparticle allows for control of the binding strength and the binding kinetics. In one embodiment, the nanoparticles can be prepared by first mixing a polyester, an activated PEG, and a biologically active agent to prepare the nanoparticles, then reacting the PEG with a bone targeting group to produce a bone targeting nanoparticle. In another embodiment, the activated PEG, for example in Formula VI, can be reacted with a bone targeting agent to produce a bone targeting PEG which can subsequently be mixed with a polyester and a biologically active agent to produce a bone targeting nanoparticle. Alternatively, an activated polyester can be reacted with a bone targeting agent, then subsequently reacted with PEG, including but not limited to another activated PEG or bone targeting PEG, and a biologically active agent. Other embodiments will become apparent by the examples below.

The composition of the invention comprises a bone targeting agent attached to nanoparticles that can strongly bind to and be retained by the hydroxyapatite surface of the bone, thereby allowing the delivery of a biologically active or therapeutic agent that can modify a cellular response. The term “modifying a cellular response” means delivering a substance (e.g., drug) that changes the way a cell would normally behave in the absence of the substance. Therapeutic or biologically active agents that could modify a cellular response include but are not limited to hormones and steroids; bioactive peptides, polypeptides, and enzymes; antisense polynucleic acids; bone growth factors such as those described in International Patent Publication WO 94/00145; cytotoxic drugs, toxins, and chemotherapeutic agents; chemoprotective and prophylatic agents including p53 inhibitors; bone marrow stimulants; and antibacterials.

The invention also provides for a method of delivering an exogenous substance to a mammal. The exogenous substance is absorbed, adsorbed, encapsulated, or chemically bonded into a biodegradable nanoparticle that bears a bone targeting agent. The exogenous substance can be any known compound or mixture, and can modify a cellular response. Preferably the exogenous substance comprises one or more drugs, proteins, nucleic acids, or mixtures thereof. The exogenous substance can also be a therapeutic or biologically active agent.

In one embodiment, the biologically active or therapeutic agent can be a chemotherapeutic agent. Chemotherapeutic agents can include adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, esperamicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, tauromustine, teniposide, thioguanine, thiotepa, Vinca alkaloids, vinblastine, vincristine, vinorelbine, paclitaxel, transplatinum, 5-fluorouracil, and the like.

The modification of cellular response can be temporary or a permanent inhibition, e.g., of p53. The gene for p53 is well known and has been studied extensively. The p53 protein is a key player in the cellular stress response mechanism. For example, in response to DNA damage the tumor suppressor protein p53 shuts down cell division or causes the cell to undergo apoptosis (programmed cell death). In this manner p53 can serve to stop tumor formation by stopping cells that have incurred malignant mutation from growing. The p53 gene is susceptible to damage and if damaged it can contribute to genetic instability and ultimately possible tumor formation. It is thought that roughly half of all cancers (including skin, breast, and colon cancers) possess mutant inactive p53 genes. On the other hand, p53 imparts sensitivity to normal tissue subjected to genotoxic stress such as radiation therapy or chemotherapy. In particular, damage to the lymphoid, hematopoietic system, intestinal epithelium, and even hair follicles contribute to collateral damage when undergoing cancer therapies and serve to limit the maximum tolerated doses of treatment.

The modification of a cellular response to impart protective activity (e.g., p53 inhibition) need not be in response to exogenous, environmental stimuli. Many processes in the body can result in cell damage, which can be inhibited by administration of the composition of the invention. For example, ischemia and ischemia/reperfusion injury can be minimized by a inhibiting cell death. Ischemia is often caused by an interruption of the supply of oxygenated blood, such as that caused by a vascular occlusion. Vascular occlusions can be caused by arteriosclerosis, trauma, surgical procedures, disease, and/or other indications. Many methods of identifying a tissue at risk of suffering ischemic damage are available. Such methods are well known to physicians who treat such conditions and include, for example, a variety of imaging techniques (e.g., radiotracer methodologies such as ^{99m}Tc-sestamibi scanning, x-ray, and MRI scanning) and physiological tests. In inhibiting cell death associated with, for example, peripheral vascular disease, the compositions of the invention can be used to direct the drug loaded nanoparticle to bone tissue adjacent to musculature suffering from or at risk of suffering from ischemia/reperfusion injury. In treating, for example, myocardial ischemia, the composition of the invention can bind to arterial calcium deposits for release of the cellular response modifying agent in the vicinity of the myocardium.

Targeted drug delivery systems utilizing bone targeting agents to delivery cell protection factors are further described in U.S. patent application Ser. No. ______ (Attorney Docket No. 224297) and U.S. Provisional Patent Application No. 60/460,289, filed Apr. 3, 2003, commonly assigned and herein incorporated by reference in their entirety.

In a preferred embodiment, the active agent is a compound of Formula I:

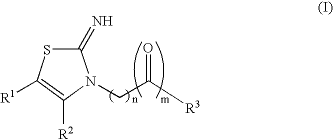

wherein m is 0 or 1, n is an integer from 1 to 4, R¹ and R² taken together form an aliphatic or aromatic carbocyclic 5- to 8-membered ring, optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties, and R³ is selected from the group consisting of a C₁-C₆ alkyl group, a C₁-C₆ alkoxy group, and a phenyl group. The alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and optionally forms a C₃-C₆ cycloalkyl when R³ is connected to the β carbon to the thiazole ring.

By “aliphatic” is meant an organic radical derived from an open straight or branched hydrocarbon chain. Examples of aliphatic moieties include, for example, alkanes, alkenes, and alkynes (e.g., C₁-C₆ alkyl, C₂-C₆ alkenyl, or C₂-C₆ alkynyl radicals, straight or branched chains).

Examples of alkyl, alkenyl, and alkynyl include, but are not limited to, methyl, ethyl, ethenyl, n-propyl, isopropyl, n-butyl, isobutyl, sec-butyl, tertiary-butyl, n-pentyl, isopentyl, n-hexyl, cis-propenyl, trans-propenyl, 2-cis-butenyl, 2-trans-butenyl, propynyl, butynyl and the like. The term “alkyl,” “alkenyl,” and “alkynyl” is also meant to include cycloalkyl, cycloalkenyl, and cycloalkynyl moieties (e.g., “C₁-C₆ alkyl” encompasses cycloalkyl, C₃-C₆ alkenyl emcompasses cycloalkenyl with rings of 3 to 6 carbons, etc.)

By “aromatic” is meant a monocyclic or polycyclic set of unsaturated carbons, e.g., phenyl. Similarly, “heteroaromatic” is a monocyclic or polycyclic set of carbons wherein one or more carbons is replaced with a nitrogen, oxygen, or sulfur atom. Examples include, but are not limited to, furyl, pyridyl, pyramidyl, quinolyl, thienyl, and thiazyl groups. It is understood that the term aromatic applies to cyclic substituents that are planar and comprise 4n+2π electrons, according to Hückel's Rule.

By “alkoxy” is meant an —OR group, wherein R is alkyl or aryl.

By “amino” is meant an —NH₂ group. By “alkylamino” is meant an —NH₂ substituted with one or two C₁-C₆ alkyl or aryl groups, e.g., monoalkyl and dialkylamino. Examples include, but are not limited to, amino, methylamino, dimethylamino, diethylamino, methylethylamino, or phenylamino.

By “alkylthio” is meant an organic radical derived from an open, straight or branched hydrocarbon chain wherein the terminus of the organic radical terminates in a —SH group (thiol group).

By “acyl” is meant a carbonyl-R group, i.e., —C(═O)—R, wherein the carbonyl is bound to an alkyl group and a heteroatom.

The biologically active compound can be a compound of Formula I, wherein m is 0, n is 2, and R³ is a one-carbon alkyl such that the three-carbon chain forms a cyclopropyl group. In other words, the biologically active compound is a compound of Formula II:

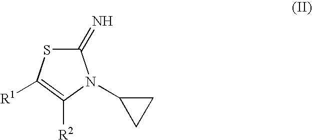

where R¹ and R² taken together form an aliphatic or aromatic carbocyclic 5- to 8-membered ring optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties.

The biologically active compound can be a compound of Formula I or Formula II, wherein R¹ and R² taken together form a 5- or 6-membered aliphatic carbocyclic ring. The 5- or 6-membered aliphatic carbocyclic ring optionally is substituted with one or more C₁-C₆ alkyl groups.

In another preferred embodiment, the biologically active compound is a compound of Formula IV:

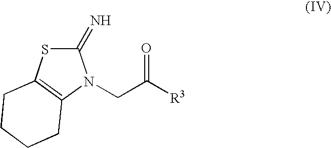

wherein R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic groups. Even more preferably, the biologically active compound is a compound of Formula V:

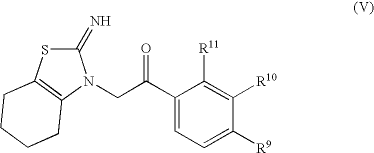

wherein R⁹, R¹⁰, and R¹¹ are each independently a hydrogen, hydroxyl, methyl, fluoro, chloro, bromo, nitro, amino, methoxy, or phenyl. Examples of the substitution around the aromatic ring include, but are not limited to, 2-, 3-, and 4-methyl, 2-, 3-, and 4-methoxy, 2-, 3-, and 4-nitro, amino, 2,4-dimethyl, 3,4-dimethyl, 2-methoxy-3-methyl, 2-methoxy-4-methyl, 3-methoxy-4-methyl, 2-methyl-3-methoxy, 2-methyl-4-methoxy, 3-methyl-4-methoxy, 2-, 3-, and 4-chloro, 2-, 3-, and 4-fluoro, 2-, 3-, and 4-hydroxy. Desirably, the biologically active compound is 2-[2-imino-4,5,6,7-tetrahydro-1,3-benzothiazol-3(2H)-yl]-1-(4-methylphenyl)-1-ethanone (i.e., pifithrin-α, shown in FIG. 11) or 2-[2-imino-4,5,6,7-tetrahydro-1,3-benzothiazol-3(2H)-yl]-1-(biphenyl)-1-ethanone.

The biologically active compound can be a compound of Formula III:

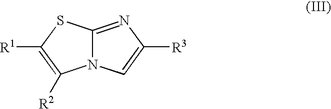

wherein R¹ and R² taken together form an aliphatic or aromatic carbocyclic 5- to 8-membered ring, optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties. R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. Preferably, the biologically active compound is 2-p-Tolyl-5,6,7,8-tetrahydro-benzo[d]imidazo[2,1-b]thiazole (i.e., pifithrin-β, shown in FIG. 12).

Pifithrin-α was recently disclosed during work based on the hypothesis that if one could block p53 protein on a temporary basis in an animal with p53 deficient tumors then one could prevent the p53 initiated cell death in the normal tissues and hence prevent many of the side effects associated with chemotherapy and/or radiation treatments. (See U.S. Pat. No. 6,593,353 and U.S. Published Patent Application 2003/0176318) A 10 micromolar concentration of pifithrin-α inhibited apoptosis induced by doxorubicin, etoposide, Taxol, cytosine arabinoside, UV light, and gamma radiation in C8 (mouse embryo fibroblasts transformed with Ela+ras) cells. To be effective, pifithrin-α needed to be present during or immediately (less than 3 hours) after exposure to UV, for example, in order to provide the protective effect. Pretreatment with removal before the stress-inducing event provide no significant protection. Pifithrin-α was also tested in two different strains of mice with the pifithrin-α being administered as a single intraperitoneal injection (2.2 mg/kg of body weight). Remarkably, this compound completely rescued both types of mice from 60% killing doses of gamma radiation (8 Gy for C57BL strain and 6 Gy for Balb/c strain). Additionally the treated animals experienced less weight-loss than controls. Importantly, in p53-null mice controls treated with radiation the pifithrin-α injections had no protective effect. Lastly, inhibition of p53 could potentially lead to tumor formation yet no tumors or pathological lesions were found in the pifithrin-α treated, gamma-irradiated survivors even after 7 months post-irradiation.

One concern with systemic administration of pifithrin-α or pifithrin-β is the potential for side effects. These types of heterocyclic compounds have known biological activities including interaction with alkaline phosphatase, glutamate transmission in epilepsy, and influencing multidrug resistance via P-glycoproteins. In addition, systemic administration of a temporary p53 inhibitor, e.g., pifithrin-α or pifithrin-β, during concurrent chemotherapy or radiation treatment would prevent cell death in the cancer cells. Thus, one major advantage of the present invention is the targeting of such molecules to the desired tissue (bone) using nanoparticles that would result in a better (e.g., reduced) side-effect profile and more effective treatment regimens.

In another preferred embodiment, the modification of a cellular response can be activation p53. Activating inactive p53 to active p53 would render cells more sensitive to chemotherapy or radiation treatment. One such low-molecular weight molecule has recently been described (Foster et al., Science, 286, 2507 (1999)) to convert mutant inactive p53 into active p53. In another preferred embodiment, the modification of a cellular response comprises stimulating bone marrow cells. Such stimulants include, but are not limited to granulocyte stimulating factors and cytokines (Bennett et al., Journal of Clinical Oncology, 17, 3676 (1999); amino boronic dipeptides such as PT100 (Foubister, V. Drug Discovery Today, 8 659, (2003)); 5-Androstenediol (Whitnall, M. H., Radiation Research, 156, 283 (2001); and the like. Cocktails of drugs could be used to both protect bone marrow cells and stimulate bone marrow cells.

The nanoparticles of the present invention can contain more than one biologically active or therapeutic agent. The two therapeutic agents could have a synergistic effect when delivered simultaneously, or a complementary effect. For example, a combination of the bone targeted-p53 inhibitor described herein with a tumor localizing small molecule p53 activator is a potent way to treat p53 mutant tumors and spare the marrow from toxicity. The nanoparticle of the present invention could also be designed to deliver the two reagents at different points in time and/or at different rates. One drug could have a higher affinity for the nanoparticle, due to, for example, hydrophobicity/hydrophilicity, acidity/basicity, or favorable enantiomer-enantiomer interactions, and therefore a slower release rate than the complementary drug.

The composition of the invention can be administered to other regions of the body containing calcium deposits for delivery of the biologically active agents. For example, a compound of the invention can be administered to an animal to inhibit cell death associated with ischemia, such as ischemia/reperfusion injury of the heart or limbs, wherein the ischemia is associated with calcium deposits in the vasculature (e.g., arterial calcification).

In another embodiment, the invention provides for a method of modifying a cellular response in a mammalian cell comprising contacting the mammalian cell with a biodegradable nanoparticle. The biodegradable nanoparticle comprising an active agent, a biodegradable polymer, and a cell targeting agent, e.g., a bone targeting agent. The contacting of the mammalian cell can be in vitro or in vivo.

The nanoparticles of the present invention will contain any acceptable ratio of components. In terms of the total weight of a nanoparticle, the active agent present in the composition can be present in 0.1-90%, preferably 1-50%, and more preferably 5-25% by weight. The biodegradable polymer can be present in 1-99%, preferably 10-90% and more preferable 25-85% by weight. The PEG may be present in 0.1-50%, preferably 1-40% and more preferably 5-25% by weight. The bone targeting agent may be present in from 0.1-50%, preferably 0.5-25% and more preferably 1-10% by weight.

The composition of the invention may be formulated in various manners, especially for administration to a mammal in, for example, therapeutic and prophylactic treatment methods. The composition for use in the inventive method comprises one or more compounds described herein and a physiologically-acceptable (e.g., pharmaceutically-acceptable) carrier. Pharmaceutically-acceptable carriers are well-known to those who are skilled in the art, as are suitable methods of administration of such compositions to a mammal. The choice of carrier will be determined in part by the particular compound within the composition, as well as by the particular method used to administer the composition. Likewise, various routes of administering a composition to a mammal are available. Although more than one route may be available, a particular route of administration may provide a more immediate and more effective response in the mammal than another route.

Ideally, the composition of the invention (e.g., a bone targeting biodegradable nanoparticle containing a therapeutic agent) is administered parenterally (e.g., subcutaneous, intramuscular, intravascular, intraspinal, intrasternal, intravenous, intrathecal, or intraarterial administration). Formulations suitable for parenteral administration are well known in the art, and include aqueous and non-aqueous, isotonic sterile injection solutions, which may contain anti-oxidants, buffers, bacteriostats, and solutes that render the formulation isotonic with the blood of the mammal, and aqueous and non-aqueous sterile suspensions that may include suspending agents, solubilizers, thickening agents, stabilizers, and preservatives. Parenteral formulations can be presented in unit-dose or multi-dose sealed containers, such as ampules and vials, and may be stored in a freeze-dried (lyophilized) condition requiring only the addition of the sterile liquid carrier, for example, water, for injections, immediately prior to use. Extemporaneous injection solutions and suspensions may be prepared from sterile powders, granules, and tablets.

In one embodiment, a composition or nanoparticles of the invention is administered directly to the area surrounding bone. While such procedures are invasive, direct administration to bone or bone marrow can provide a more immediate effect than, for instance, intravenous administration. A surgical procedure similar to that for aspirating bone marrow can be performed to administer the inventive composition directly to bone marrow. At least a portion of the inventive composition remains attached to the bone tissue via the bone targeting agent, which creates a sustained release mechanism of the biologically active agent to the bone marrow.

While not particularly preferred, the composition can be introduced into a mammal via oral, nasal, topical, rectal, or vaginal administration. Formulations suitable for oral administration can comprise powders, liquid solutions, such as an effective amount of the inventive compound dissolved in diluents, such as water, saline, or orange juice, as well as capsules, sachets or tablets, each containing a predetermined amount of the active ingredient. Oral formulations can be presented as solids or granules; solutions or suspensions in an aqueous liquid; and oil-in-water emulsions or water-in-oil emulsions. Tablet forms may include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

Aerosol formulations to be administered via inhalation can be incorporated into pressurized acceptable propellants, such as-dichlorodifluoromethane, propane, nitrogen, and the like. Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, gels, and the like containing, in addition to the active ingredient, such carriers as are known in the art.

Formulations for rectal administration commonly comprise a suppository with a suitable base comprising, for example, cocoa butter or a salicylate. Formulations for vaginal delivery can comprise, for example, pessaries, tampons, creams, gels, pastes, foams, or spray formulas containing, in addition to the active ingredient, such carriers as are known in the art to be appropriate.

The appropriate dose of the composition administered to a mammal in accordance with the inventive method should be sufficient to effect the desired response in the mammal over a reasonable time frame. Dosage will depend upon a variety of factors, including the age, species, and size of the mammal. Dosage also depends on the particular therapeutic agent, nanoparticle formulation, and bone targeting agent that are employed. The size of the dose also will be determined by the route, timing, and frequency of administration as well as the existence, nature, and extent of any adverse side effects that might accompany administration and the desired physiological effect. Some situations, such as exposure of a mammal to multiple rounds of chemotherapy or radiation therapy, may require prolonged treatment involving multiple administrations. The actual dose of the inventive composition can range from about 0.05 milligrams per kilogram of body mass to about 100 milligrams per kilogram of body mass.

The inventive composition can be packaged in unit dosage form, i.e., physically discrete units suitable as unitary dosages for a mammal, each unit containing a predetermined quantity of the composition or nanoparticles calculated in an amount sufficient to produce the desired level of cellular response modification in association with a pharmaceutically acceptable diluent, carrier, or vehicle. Unit dosage forms can be incorporated into a kit, wherein the composition of the invention is provided in combination with a physiologically-acceptable carrier and instructions for administration to a mammal.

The following examples further illustrate the invention but, of course, should not be construed as in any way limiting its scope.

Materials and Methods: HPLC analysis was performed on a Shimadzu LCMS-2010 and employed a flow rate of 3 ml/min and a starting B concentration of 5%. The B solvent was linearly ramped to 95% concentration at 5.0 minutes, held at 95% until 6.0 minutes, then linearly ramped back down to 5% at 6.5 minutes, where it remains until the end of the run at 7.5 minutes. In addition to an electrospray mass spectrometer, the LC detection consisted of 3 channels: UV absorbance at 254 nm, UV absorbance at 214 nm, and evaporative light scattering (Alltech ELSD 2000). The evaporative light scattering detector was run at 50 C with a nitrogen flow of 1.5 liters per minute. The CDL and block temperatures of the Shimadzu LCMS-2010 were both 300° C., and the nitrogen nebulizer gas flow was 4.5 L./min. Positive and negative mass spectra were detected from 50 to 2000 m/z. The column was a YMC CombiScreen ODS-AQ, S-5μ particle size, 50 mm long with a 4.6 mm I.D. Mobile phase A was made using HPLC grade B&J water with 0.1% (v/v) HOAc added and mobile phase B was HPLC grade B&J acetonitrile with 0.1% (v/v) HOAc added. This system gives a retention time of 1.50 to 1.60 minutes for a standard commercially available material (4-hydroxyphenylacetic acid; Aldrich Catalog H5000-4; m.p. 149-151 C) used as a reference standard.

Gradient Preparative HPLC was performed on a Shimadzu system composed of two LC-8A pumps connected to a SIL-10A autosampler and eluting over a reverse phase column (YMC, cat CCAQSOSO52OWT; ODS-AQ CombiPrep, 20 mm×50 mm) and then passing through an MRA variable volume splitter; the smaller stream was then made up to 3 mL/minute using a LC-10ADVP make-up pump (MeOH) and the eluent passed through a variable two channel wavelength UV detector and then split roughly 6:1 to an evaporative light scattering detector (run at 50 C with a nitrogen flow of 1.5 liters per minute) and a Shimadzu 2010 Mass detector; the larger stream from the MRA splitter then flowed to a Gilson 215 liquid handler serving as a fraction collector triggered by mass, UV absorbance, or ELS peak size. Different gradients were run always starting with the more aqueous solvent A and ramping up to various concentrations of B. Mobile phase A was made using HPLC grade B&J water with 0.1% (v/v) HOAc added and mobile phase B was HPLC grade B&J acetonitrile with 0.1% (v/v) HOAc added.

### EXAMPLE 1

This example illustrates a method of preparing pifithrin-α.

A small sample (<500 mgs) of pifithrin-α was prepared according to literature methods as shown in FIG. 14 (see, for example, International Patent Publication WO 00/44364; Tasaka et al., J. Heterocyclic Chem., 34, 1763 (1997); and Andreani et al., J. Med. Chem., 38, 1090 (1995)). Referring to FIG. 14, the 2-aminothiazole (Q) was prepared by reacting chlorocyclohexanone (O), thiourea (P), N-bromosuccinimide, and benzoyl peroxide in toluene heated to reflux overnight. Then the solvent was removed, and the solid was recrystallized from hexane. This sample (Q) was then dissolved with a slight excess of commercially available p-methylphenacyl bromide (R) in toluene and then stirred for 48 hours at room temperature, at which time pifithrin-α precipitated out of solution as the HBr salt. Pifithrin-α.HBr was converted into the pifithrin-α free base by neutralization with 1M NaOH and subsequent extraction with chloroform.

### EXAMPLE 2

This example illustrates a method for preparing pifithrin-β in one reaction.

A solution of 2-chlorocyclohexanone in toluene was treated with 1.1 equiv of thiourea and 1.1 equiv of triethylamine. The mixture was heated at 95° C. overnight. To this solution was added 1.3 equiv of 2-bromo-4′-methylacetophenone, and the mixture stirred overnight to produce a tan solid. The solid was filtered and washed with toluene. The solid was taken up in chloroform and 10% (wt/wt) potassium bicarbonate and stirred 5 minutes, resulting in dissolution of the solid. The layers were separated, the aqueous layer extracted with more chloroform, and the combined organic layers were washed with 5% (wt/wt) potassium carbonate. The organic solution was dried over sodium sulfate and the solvent removed to give a brown solid. The solid was subjected to silica gel column chromatography using 80/20 dichloromethane/methanol. Fractions were recovered containing pifithrin α and pifithrin β. These fractions were dissolved in dioxane and heated at 90° C. overnight. After cooling and filtration, a yellow solid of pifithrin-β was obtained in 13.1% overall yield. This material characterized by LC-MS to give a retention time of 3.9 minutes and showed the desired mass of [M+H]+=269 m/z and was identical in retention time and mass spectral ionization to commercially available material (Cal-BioChem; catalog number 506134).

### EXAMPLE 3

This example illustrates a method for preparing a PEG-modified bone targeting agent.

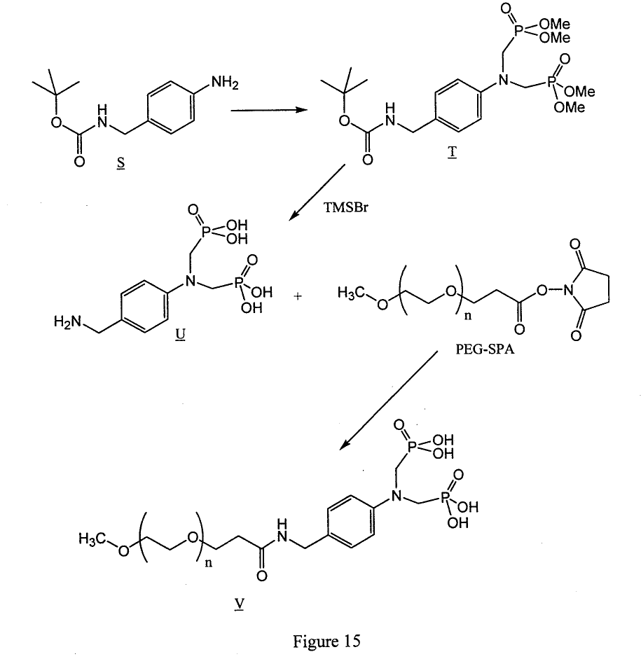

A solution of 500 mg 4-[(N-Boc)aminomethyl]aniline (S in FIG. 15) in 10 mL dioxane was treated with paraformaldehye (400 mol %, 270 mg) and trimethylphosphite (400 mol %, 1.12 g). The mixture was heated to 95° C. overnight. More paraformaldehyde (270 mg) and trimethylphosphite (1.12 g) were added and the mixture was heated at 95° C. overnight again. The solution was cooled, taken up in chloroform (20 mL) and washed with saturated sodium chloride (20 mL) and water (20 mL). The organics were dried over sodium sulfate and the solvent and excess trimethylphosphite removed via rotary evaporation at 80° C. to provide 1.723 g of a clear oil. The presence of the diphosphonate (T) was confirmed by electrospray HPLC-MS showing a retention time of t_{R}=2.9 minutes and a mass of 467 m/z [M+H]+ and 489 m/z [M+Na]+ found for the desired mass [M=C₁₈H₃₂N₂O₈P₂].

A solution of 870 mg diphosphate (T) in 10 mL dichloromethane was treated with bromotrimethylsilane (690 mol %, 1.97 g). The solution was stirred overnight. Methanol (10 mL) was added and the solution was stirred 15 min and then concentrated to provide 1.12 g of an orange oil. The presence of the diphosphonic acid (U) was confirmed by electrospray LC-MS. The retention time using this gradient was found to be t_{R}=0.85 minutes and the mass spec for the desired product [M=C₉H₁₆N₂O₆P₂] found at the expected m/z 309 [M−H]⁻ operating in the negative mode.

A 50 mg portion of the diphosphonic acid (U) was solubilized in 200 uL of water and adjusted to pH=8.1 using NaOH and then treated with 100 mg (0.125 equivalents) of PEG-SPA (Shearwater; 5,000 molecular weight) and 100 μL of water, then allowed to stir for 24 hours. Analysis by HPLC showed that the starting PEG-SPA peak had largely disappeared (retention time 2.97 minutes) and was replaced by a UV-absorbing peak at about 3.75 minutes. The sample was then purified on prep HPLC to give 44.6 mg of the bone targeting aminodiphosphonic acid (V) coupled to a 5,000 molecular weight PEG) having a retention time of 3.75 minutes and exhibiting UV activity at 254 nm and exhibiting a mass spectrum supportive of a polymeric structure.

### EXAMPLE 4

This example illustrates a method for preparing a PEG-Fluorescein complex.

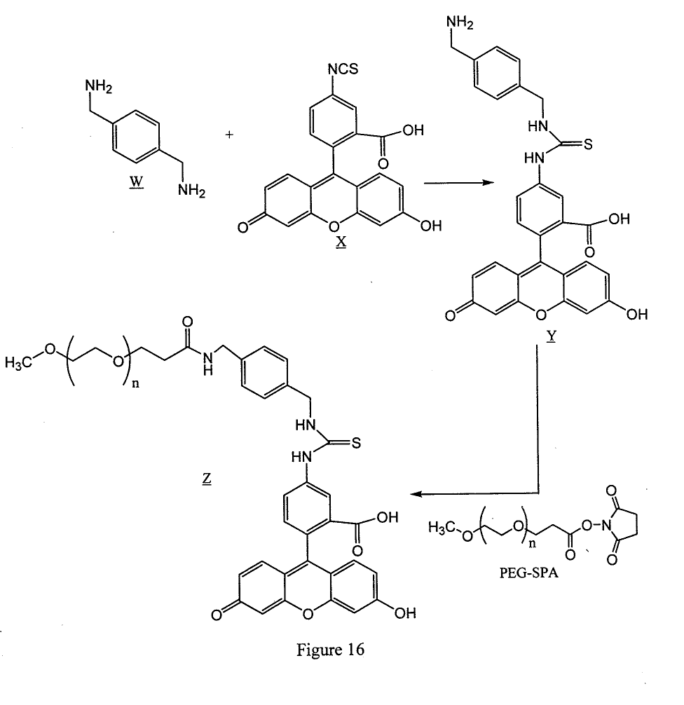

A solution of 210 mg p-xylylenediamine (W in FIG. 16) in 6 mL methanol was treated with a fluorescein isothiocynate (X, FTIC Isomer 1—Calbiochem, catalog number 34321) in small portions over 1 hr. During the addition, a red-brown solid was produced. The mixture was stirred overnight, filtered, and washed with methanol (2 mL). The filtrate was concentrated to a red oil which solidified upon drying. A portion of this solid was purified via prep HPLC to provide 53.4 mg of an orange solid. The product (Y) was confirmed by electrospray LC-MS giving a retention time t_{R}=2.1 minutes and MS [M=C₂₉H₂₃N₃O₅S] m/z 526 (MH⁺).

A 10 mg (19 μMoles) sample was dissolved in 500 μL of DMF and treated all at once with 50 mg (10 μMoles) of mPEG-SPA (5000 molecular weight) and 10 μL of triethylamine and allowed to stir for 9 hours yielding an orange suspension. An HPLC of the sample diluted in methanol indicated that about half of the starting material (retention time 2.1 minutes) had been converted to a UV containing peak with longer retention time (3.1 minutes). The mixture was then purified by prep HPLC to give 43.6 mg of bright orange solid with a retention time of 3.12 minutes and having strong UV peak absorption on both the 254 nm and 214 nm UV detectors and a mass spec pattern indicative of polymeric species (Z).

### EXAMPLE 5

This example illustrates a method for preparing a diphosphonic acid bone targeting agent.

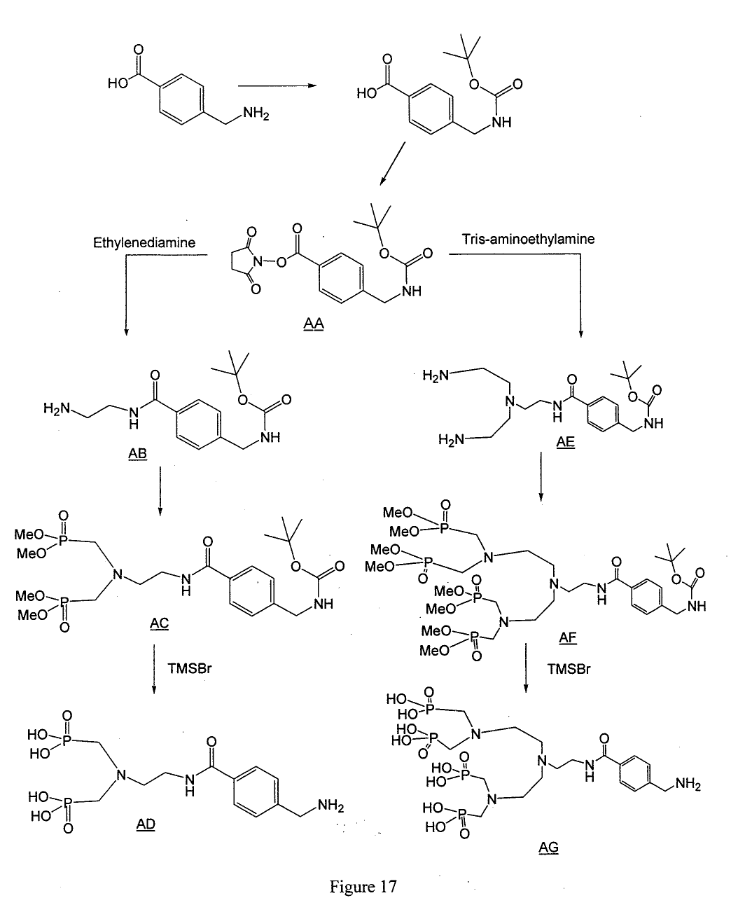

A solution of sodium hydroxide was prepared by dissolving 0.64 g of NaOH in 20 mL of water. A 2.0 g portion of 4-aminomethyl benzoic acid (Aldrich) was added and then 3.18 g of Boc-anhydride (Aldrich) added in one portion. (See FIG. 17.) The suspension was stirred overnight and after 16 hours carefully acidified to pH=2 by the addition of 15 mL of 2N hydrochloric acid. The white solid was filtered and washed with a small amount of water and dried under vacuum to yield 2.9997 g of 4-[N-Boc-aminomethyl]benzoic acid. The compound was >95% pure by HPLC-MS and showed a retention time of 2.901 minutes with the expected mass spectrum in the negative mode ([M−H]⁻=250 m/z; for presence of the desired compound of formula C13H17NO4).

A solution of 1.29 g of the Boc-protected aminomethylbenzoic acid in 15 mL tetrahydrofuran was treated with N-hydroxysuccinimde (623 mg) and 5.4 mL of a 1.0 M solution of 1,3-dicyclohexylcarbodiimide in dichloromethane. The mixture was stirred overnight, then the white precipitate was filtered and the supernatant concentrated to provide 1.89 g of the activated ester AA as a white solid.

A 500 mg portion of the activated ester (AA) was dissolved in 5 mL of dry tetrahydrofuran and treated with 1.002 mL (10 equivalents) of ethylenediamine with vigorous stirring. An immediate white precipitate formed. The suspension was stirred for 2 hours, then the solid filtered and washed with THF and dried to give 0.8728 g of white solid. This material was characterized by HPLC which showed complete conversion of starting activated ester to the desired product (AB) with a retention time of 1.608 minutes and the expected mass spectrum ([M+H]+=294 for C15H23N3O3-H+).

A solution of 872 mg of the amine (AB) in 10 mL dioxane was treated with paraformaldehyde (535 mg) and trimethylphosphite (2.21 g). The mixture was heated at 100° C. overnight and then the solvent removed by rotary evaporation at 80° C. to give a brown solid. Chloroform (25 mL) was added and the solution was washed with water (15 mL). The organics were dried over sodium sulfate and the solvent removed to provide 241 mg of a yellow semi-solid. This was purified via LC to provide 58.8 mg of diphosphonate ester (AC) material. The compound was confirmed by electrospray LC-MS using method A; t_{R}=2.6 min. MS [M=C₂₁H₃₇N₃O₉P₂] m/z 538 (MH⁺), 560 (MNa⁺).

A solution of 54.6 mg of diphosphonate (AC) in 1 mL dichloromethane was treated with bromotrimethylsilane (156 mg). The mixture was stirred overnight. Ethanol (0.5 mL) and water (3 drops) were added and it was stirred 1 hr and then the volatiles were removed and the material dried under vacuum. This was taken up in water (1 mL) and lyophilized to provide 59 mg of a tan solid. The presence of the free amine diphosphonic acid (AD) was confirmed by electrospray LC-MS using method B; t_{R}=0.4 min. MS [M=C₁₂H₂₁N₃O₇P₂] m/z 380 (M−H⁻), 382 (MH⁺), 404 (MNa⁺)

The amine diphosphonate can be converted to a PEG-modified bone targeting agent by treating 50 mg with 650 mg of mPEG-SPA in water, then freeze-drying to isolate the PEG-aminodisphosphonic acid bone targeting agent.

### EXAMPLE 6

This example illustrates a method for preparing a tetraphosphonic acid bone targeting agent.

To a solution of 2.1 g tris-(2-aminoethyl)amine (see FIG. 17) in 20 mL tetrahydrofuran was added dropwise a solution of 1.0 g of the activated ester (AA) in 20 mL tetrahydrofuran over a period of 40 minutes. The mixture was stirred overnight resulting in a precipitate that was filtered and concentrated via rotary evaporation to provide 2.10 g of a yellow oil. The presence of the diamine (AE) was confirmed by electrospray LC-MS using method A; t_{R}=1.4 min. MS [M=C₁₉H₃₃N₅O₃] m/z 380 (MH⁺), 402 (MNa⁺).

A solution of 2.08 g diamine (AE) in dioxane (20 mL) was treated with paraformaldehyde (1.50 g) and dimethylphosphite (6.85 g). The mixture was stirred at 90° C. overnight and the solvent removed via rotary evaporation at 70° C. Dichloromethane (50 mL) was added and it was washed with saturated sodium chloride (25 mL) and water (25 mL). The organics were dried over sodium sulfate and the solvent removed. The residue was purified via LC to provide 123.8 mg of a yellow oil. The presence of the tetraphosphonate diamine (AF) was confirmed by electrospray LC-MS using method D; t_{R}=2.2 min. MS [M=C₃₁H₆₁N₅O₁₅P₄] m/z 868 (MH⁺).

A solution of 111.1 mg of the tetraphosphonate diamine (AF) in 1 mL dichloromethane was treated with 194 mg of bromotrimethylsilane. After 5 hr, methanol (1 mL) was added, the mixture was stirred for 1 hr, and the solvent was removed to provide 113.9 mg of a tan solid. The presence of the free amine tetraphosphonic acid (AG) was confirmed by electrospray LC-MS using method B; t_{R}=1.0 min. MS [M=C₁₈H₃₇N₅O₁₃P₄] m/z 328 [(M+2H/2)²⁺)], 656 (MH⁺).

The amine tetraphosphonate can be converted to a PEG-modified bone targeting agent by treating 50 mg with 400 mg of mPEG-SPA in water, then freeze-drying to isolate the product.

### EXAMPLE 7

The example illustrates a method for preparing nanoparticles in accordance with an embodiment of the invention.

For the following examples PLGA is polylactic polyglycolic acid copolymer (50/50) of about 13,000 molecular weight with uncapped ends from Alkermes Inc. The PEG is polyethylene glycol of 5,000 molecular weight from Nektar, Huntsville Ala. (formerly Shearwater Corporation). BSA is bovine serum albumin. Alendronate is 4-Amino-1-hydroxybutylidine-1,1-bisphosphonate, sodium trihydrate, from Calbiochem catalog number 126855. mPEG-SPA is a 5000 molecular weight polyethylene glycol with a methyl ether on one end and a succinimidyl propionate activated ester on the other end; catalog 2M4M0H01 from Nektar, Huntsville Ala. (formerly Shearwater Corporation). mPEG-NHS is also a 5000 molecular weight polyethyleneglycol from Nektar, with a methyl ether on one end and an N-hydroxylsuccininmidyl carbonate on the other end. The PEG-Alendronate complex was prepared by mixing a solution of 600 mg of mPEG-SPA (MW 5000) and 6 mg of alendronate in 20 ml of water and stirring for 30 minutes, then freeze-drying the solution overnight.

The weight percent of pifithrin α or β in the nanoparticles was determined by weighing a 2, 4, or 6 mg sample of each batch into a 1 dram vial. 1.00 ml methylene chloride was added, and the vial placed on an orbital shaking at 200 rpm for two hours, then stirred with a magnetic stir bar at high speeds for approximately five minutes in order to completely dissolve the nanoparticles. When the solution was visually clear, 2.00 ml MeOH was added, causing cloudiness to appear, and 0.4 ml of this suspension was pipetted in the bottom half of a Whatman MiniUniPrep vial filter device. The top half was affixed to filter out the nanoparticles, and the resulting filtered solution was run on an HPLC-MS using a reverse phase column with an injection volume of 10 μl. A calibration curve using concentrations of pifithrin α or pifithrin β of 100, 500, and 1000 μM was used for quantitation using the 254 nm absorbance for the cell protection factor peak.

The experimental details and analysis for the nanoparticle preparations are set forth in Table 1. In a typical procedure, the organic solution (shown in column 2 of Table 1) was sonicated for 30-120 seconds, then added to the aqueous layer (shown in column 3) contained within a 20 ml scintillation vial. Deionized water was used to prepare the aqueous solutions and water washes. This solution was sonicated for an additional 60 seconds, then transferred to a 125 ml Erlenmeyer flask and set to stir at 250 to 500 rpm under vacuum for 45 minutes (shown in column 4, “Experimental Conditions”). Vacuum was typically achieved at about 3 to 10 inches of mercury. After 45 minutes, the contents were poured into a centrifuge tube and the flask rinsed with 10 ml of water. The particles were spun down at 18000 rpm for 10 minutes and the supernatant pipetted out. A 10 ml wash solution of a 1 mg/ml BSA solution was added to the centrifuge tube, the sample was sonicated until the particles were completely resuspended, the sample was spun down at 18000 rpm for 10 to 15 minutes, and the supernatant was removed by pipet. This wash/spin cycle was repeated a total of three times. After the third cycle, the supernatant was removed, the particles were frozen overnight, and placed on a freeze dryer for 24 hours. Analytical results are reported in Table 1 for each preparation.

TABLE 1

| | | | | | Median | | | |
|---|---|---|---|---|---|---|---|---|
| Ex- | | | Experimental | mgs | Diameter | Weight % | Weight % | Relative |
| ample | Organic Solution | Aqueous Solution | Conditions | isolated | (nm) | pifithrin-β | pifithrin-α | Polydispersity |
| 7.1 | 100 mg PLGA in 2.5 ml acetone | 10 ml of 1 mg/ml | Sonicate 30 sec | 92.5 | 1080 | 3.4 | NA | 8 |
| | 1 ml of 10 mg/ml pifithrin-β in | BSA in water | Stir at 250 rpm | | | | | |
| | methanol | | under vacuum | | | | | |
| 7.2 | 100 mg PLGA in 2.5 ml acetone | 10 ml of 1 mg/ml | Sonicate 30 sec | 89 | 402 | 3.1 | NA | 9 |
| | 1 ml of 10 mg/ml pifithrin-β in | BSA in water | Stir at 250 rpm | | | | | |
| | methanol | | under vacuum | | | | | |
| 7.3 | 100 mg PLGA in 3.5 ml acetone | 10 ml of 1 mg/ml | Sonicate 30 sec | 87 | >3000 | 5.3 | NA | — |
| | 1 ml of 10 mg/ml pifithrin-β in | BSA in water | Stir at 250 rpm | | | | | |
| | methanol | | under vacuum | | | | | |
| 7.4 | 100 mg PLGA in 3.5 ml acetone | 10 ml of 10 mg/ml | Sonicate 30 sec | 97.9 | 628 | 10.2 | NA | 7 |
| | 1 ml of 10 mg/ml pifithrin-β in | BSA in water | Stir at 500 rpm | | | | | |
| | methanol | | under vacuum | | | | | |
| 7.5 | 100 mg PLGA in 2.0 ml acetone | 10 ml of 1 mg/ml | Sonicate 30 sec | 82.6 | 210 | 11 | NA | 4 |
| | 1 ml of 10 mg/ml pifithrin-β in | BSA in water | Stir at 500 rpm | | | | | |
| | methanol | | under vacuum | | | | | |
| 7.6 | 100 mg PLGA in 2.0 ml acetone | 10 ml of 1 mg/ml | Sonicate 30 sec | 90.4 | 234 | 4.7 | NA | 5 |
| | 1 ml of 10 mg/ml pifithrin-β in | BSA in water | Stir at 500 rpm | | | | | |
| | methanol | | under vacuum | | | | | |
| 7.7 | 100 mg PLGA in 2.5 ml acetone | 10 ml of 1 mg/ml | Sonicate 30 sec | 92.7 | 306 | 12.5 | NA | 5 |
| | 10 mg pifithrin-β | BSA in water | Stir at 250 rpm | | | | | |
| | 1.5 ml methanol | | under vacuum | | | | | |
| 7.8 | 100 mg PLGA in 3.5 ml acetone | 10 ml of 10 mg/ml | Sonicate 30 sec | 106.3 | 225 | 8.9 | NA | 3 |
| | 10 mg pifithrin-β | BSA in water | Stir at 500 rpm | | | | | |
| | 1.5 ml methanol | | under vacuum | | | | | |
| 7.9 | 100 mg PLGA in 2.0 ml acetone | 10 ml of 10 mg/ml | Sonicate 60 sec | 92.1 | 208 | 10.9 | NA | 2 |
| | 10 mg PEG-alendronate complex | BSA in water | Stir at 500 rpm | | | | | |
| | 1 ml of 10 mg/ml pifithrin-β in | 10 ml water | under vacuum | | | | | |
| | methanol | | | | | | | |
| 7.10 | 100 mg PLGA in 2.0 ml acetone | 10 ml of 10 mg/ml | Sonicate 120 sec | 103.3 | 301 | 9.6 | NA | 4 |
| | 10 mg PEG-alendronate complex | BSA in water | Stir at 500 rpm | | | | | |
| | 20 mg pifithrin-β | 10 ml water | under vacuum | | | | | |
| 7.11 | 100 mg PLGA in 4.0 ml acetone | 10 ml of 10 mg/ml | Sonicate 60 sec | 121.3 | 187 | 16.2 | NA | 4 |
| | 10 mg PEG-alendronate complex | BSA in water | Stir at 500 rpm | | | | | |
| | 2 ml of 10 mg/ml pifithrin-β in | 10 ml water | under vacuum | | | | | |
| | methanol | | | | | | | |
| 7.12 | 100 mg PLGA in 4.0 ml acetone | 10 ml of 10 mg/ml | Sonicate 60 sec | 123.2 | 273 | 8.2 | NA | 5 |
| | 10 mg PEG-alendronate complex | BSA in water | Stir at 500 rpm | | | | | |
| | 2 ml of 10 mg/ml pifithrin-β in | 10 ml water | under vacuum | | | | | |
| | methanol | | | | | | | |
| 7.13 | 100 mg PLGA in 2.0 ml acetone | 15 ml of 10 mg/ml | Sonicate 60 sec | 92.3 | 274 | 3.8 | NA | 4 |
| | 10 mg PEG-alendronate complex | BSA in water | Stir at 500 rpm | | | | | |
| | 10 mg pifithrin-β | 10 ml water | under vacuum | | | | | |
| | 1 ml methanol | | | | | | | |
| 7.14 | 100 mg PLGA in 5.0 ml acetone | 10 ml of 1 mg/ml | Sonicate 60 sec | 104.7 | 286 | 7.2 | NA | 8 |
| | 10 mg PEG-alendronate complex | BSA in water | Stir at 500 rpm | | | | | |
| | 1 ml of 10 mg/ml pifithrin-β in | 10 ml water | under vacuum | | | | | |
| | methanol | | | | | | | |
| 7.15 | 100 mg PLGA in 2.0 ml acetone | 10 ml of 10 mg/ml | Sonicate 60 sec | 103.2 | 293 | 7.5 | NA | 6 |
| | 6.6 mg PEG-alendronate complex | BSA in water | Stir at 500 rpm | | | | | |
| | 10 mg pifithrin-β | 10 ml water | under vacuum | | | | | |
| | 1 ml methanol | | | | | | | |
| 7.16 | 100 mg PLGA in 2.0 ml acetone | 10 ml of 10 mg/ml | Sonicate 60 sec | 103.8 | 304 | 8.3 | NA | 5 |
| | 20 mg PEG-alendronate complex | BSA in water | Stir at 500 rpm | | | | | |
| | 20 mg pifithrin-β | 10 ml water | under vacuum | | | | | |
| | 1 ml methanol | | | | | | | |
| 7.17 | 100 mg PLGA in 2.0 ml acetone | 10 ml of 10 mg/ml | Sonicate 60 sec | 101.4 | 238 | 2.9 | NA | 2 |
| | 10 mg PEG-alendronate complex | BSA in water | Stir at 500 rpm | | | | | |
| | 10 mg pifithrin-β | 10 ml water | under vacuum | | | | | |
| | 1 ml methanol | | | | | | | |
| 7.18 | 100 mg PLGA in 2.0 ml acetone | 10 ml of 10 mg/ml | Sonicate 60 sec | 101.4 | 244 | 4.2 | NA | 3 |
| | 15 mg PEG-alendronate complex | BSA in water | Stir at 500 rpm | | | | | |
| | 10 mg pifithrin-β | 10 ml water | under vacuum | | | | | |
| | 1 ml methanol | | | | | | | |
| 7.19 | 100 mg PLGA in 2.0 ml acetone | 10 ml of 10 mg/ml | Sonicate 60 sec | 104.2 | — | 5.3 | NA | — |
| | 20 mg PEG-SPA | BSA in water | Stir at 500 rpm | | | | | |
| | 1 ml of 10 mg/ml pifithrin-β | 2.7 ml alendronate | under vacuum | | | | | |
| 7.20 | 100 mg PLGA in 2.0 ml acetone | 10 ml of 10 mg/ml | Sonicate 60 sec | 102 | 248 | 3.9 | 0.3 | 5 |
| | 10 mg pifithrin-α | BSA in water | Stir at 500 rpm | | | | | |
| | 1 ml methanol | 10 ml water | under vacuum | | | | | |
| 7.21 | 100 mg PLGA in 2.0 ml acetone | 10 ml of 10 mg/ml | Sonicate 60 sec | 107.7 | 298 | 7.2 | 0.5 | 6 |
| | 20 mg pifithrin-α | BSA in water | Stir at 500 rpm | | | | | |
| | 1 ml methanol | 10 ml water | under vacuum | | | | | |
| 7.22 | 100 mg PLGA in 2.0 ml acetone | 10 ml of 10 mg/ml | Sonicate 60 sec | 103.9 | 247 | 6.4 | 0.3 | 5 |
| | 10 mg PEG-SPA | BSA in water | Stir at 500 rpm | | | | | |
| | 10 mg pifithrin-α | 10 ml water | under vacuum | | | | | |
| | 1 ml methanol | | | | | | | |
| 7.23 | 100 mg PLGA in 2.0 ml acetone | 10 ml of 10 mg/ml | Sonicate 60 sec | 98 | 240 | 6.7 | 0.2 | 5 |
| | 10 mg PEG-Alendronate complex | BSA in water | Stir at 500 rpm | | | | | |
| | 10 mg pifithrin-α | 10 ml water | under vacuum | | | | | |
| | 1 ml methanol | | | | | | | |
| 7.24 | 100 mg PLGA in 2.0 ml acetone | 10 ml of 10 mg/ml | Sonicate 60 sec | 103.9 | 314 | 5.9 | 0.2 | 7 |
| | 20 mg PEG-Alendronate complex | BSA in water | Stir at 500 rpm | | | | | |
| | 10 mg pifithrin-α | 10 ml water | under vacuum | | | | | |
| | 1 ml methanol | | | | | | | |
| 7.25 | 100 mg PLGA in 2.0 ml acetone | 10 ml of 10 mg/ml | Sonicate 60 sec | 105.1 | — | 6 | 0.3 | — |
| | 20 mg PEG-NHS | BSA in water | Stir at 500 rpm | | | | | |
| | 2 mg Fluorescein compound (Y) | 10 ml water | under vacuum | | | | | |
| | 10 mg pifithrin-α | | | | | | | |
| | 1 ml methanol | | | | | | | |
| 7.26 | 100 mg PLGA in 2.0 ml acetone | 10 ml of 10 mg/ml | Sonicate 60 sec | 104.2 | 933 | 5.6 | NA | 9 |
| | 10 mg PEG-Fluorescein complex | BSA in water | Stir at 500 rpm | | | | | |
| | (Z) from Example 4 | 10 ml water | under vacuum | | | | | |
| | 10 mg pifithrin-β | | | | | | | |
| | 1 ml methanol | | | | | | | |
| 7.27 | 100 mg PLGA in 2.0 ml acetone | 10 ml of 10 mg/ml | Sonicate 60 sec, stir | 109.4 | 682 | 7.2 | NA | 9 |
| | 10 mg PEG-aminomethylaniline | BSA in water | at 500 rpm under | | | | | |
| | bisphosphonate (V) Example 3 | 10 ml water | vacuum | | | | | |
| | 10 mg pifithrin-β | | | | | | | |
| | 1 ml methanol | | | | | | | |
| 7.28 | 100 mg PLGA in 4.0 ml acetone | 10 ml of 10 mg/ml | Sonicate 60 sec | 100.9 | 256 | 13.6 | NA | 3 |
| | 25 mg PEG-Alendronate complex | BSA in water | Stir at 500 rpm | | | | | |
| | 10 mg pifithrin-β | 10 ml water | under vacuum | | | | | |
| | 1 ml methanol | | | | | | | |
| 7.29 | 100 mg PLGA in 2.0 ml acetone | 10 ml of 10 mg/ml | Sonicate 60 sec | 102.3 | 330 | 3.9 | 0.2 | 7 |
| | 20 mg PEG-Alendronate complex | BSA in water | Stir at 500 rpm | | | | | |
| | 10 mg pifithrin-β | 10 ml water | under vacuum | | | | | |
| | 1 ml methanol | | | | | | | |
| 7.30 | 100 mg PLGA in 3.0 ml acetone | 10 ml of 10 mg/ml | Sonicate 60 sec | 101.8 | 300 | 5.9 | NA | 6 |
| | 10 mg PEG-aminomethylaniline | BSA in water | Stir at 500 rpm | | | | | |
| | bisphosphonate (V) | 10 ml water | under vacuum | | | | | |
| | 10 mg pifithrin-β | | | | | | | |
| | 1 ml methanol | | | | | | | |
| 7.31 | 100 mg PLGA in 4.0 ml acetone | 10 ml of 10 mg/ml | Sonicate 60 sec | — | — | — | NA | — |
| | 12.3 mg of PEG-Alendronate | BSA in water | Stir at 500 rpm | | | | | |
| | complex | 10 ml water | under vacuum | | | | | |
| | 1 ml of 10 mg/ml pifithrin-β in | | | | | | | |
| | methanol | | | | | | | |

*NA—not applicable

### EXAMPLE 8

This example illustrates the binding of nanoparticles in accordance with an embodiment of the invention to hydroxyapatite.

Nanoparticles selected from examples 7.9, 7.10, 7.11, 7.12, 7.13, and 7.31, above were assayed for hydroxyapatite binding. For each sample, a nominal 7.5 mg of each nanoparticle preparation was weighed and 1.5 ml Tris-buffered saline (50 mM 2-amino-2-(hydroxymethyl)-1,3-propanediol and 150 mM NaCl, pH 7.4) was added, for a concentration 5.0 mg/ml. The samples were then subjected to ultrasonication for one minute to form a uniform suspension of particles.

A 10.0 mg/ml suspension of hydroxyapatite was prepared by weighing 80 μm hydroxyapatite particles (Bio-Rad MacroPrep Ceramic Hydroxyapatite Type I 80 μm—Catalog No. 185-8000) and diluting with Tris-buffered saline. The hydroxyapatite particles settle quickly, so the suspension is stirred on a Thermolyne magnetic stirplate at the slowest possible speed to achieve uniformity, and sampling by pipette is performed quickly to minimize fragmentation of the hydroxyapatite particles.

Controls (100% value) were run by mixing 200 μl of the each nanoparticle sample and 200 μl of the Tris-buffered saline. Samples were prepared by mixing 200 μl of the each nanoparticle sample and 200 μl of the hydroxyapatite suspension in a 1.5 ml vial and shaking on an orbital shaker at 200 rpm for 1 hour. Each experimental cell was run in triplicate. After shaking, the hydroxyapatite particles were allowed to settle for 5 minutes and about 300 μl of the supernatant was transferred by Pasteur pipette to the bottom half of a Whatman MiniUniPrep vial. The top was affixed to filter out nanoparticles (greater than 0.2 μm), and the resulting solution was then run on the Shimadzu LCMS-2010 instrument using an injection volume of 10 μl. The UV absorbance signal at 254 nm at 2.95 minutes retention time, diagnostic of pifithrin-β, was used to calculate the percentage of pifithrin remaining in the supernatant after exposure to hydroxyapatite.

The percent reduction of pifithrin-β in the supernatant in the presence of hydroxyapatite compared to the supernatant in the absence of hydroxyapatite was used as an indicator of the amount of nanoparticles bound to the hydroxyapatite. The results are presented below:

TABLE 2

| Sample | % of pifithrin-β remaining after exposure to |
|---|---|
| Number | hydroxyapatite |
| 7.9 | 60.3 ± 7.9 |
| 7.10 | 63.0 ± 6.0 |
| 7.11 | 85.8 ± 2.1 |
| 7.12 | 93.4 ± 2.9 |
| 7.13 | 88.7 ± 21.6 |
| 7.31 | 86.0 ± 0.4 |

### EXAMPLE 9

This example illustrates the binding of nanoparticles to hydroxyapatite.

Nanoparticle samples selected from examples 7.25, 7.26, 7.27, and 7.30 listed above were subjected to the hydroxyapatite binding assay as described above. The assay showed that there was a 16% reduction of nanoparticles from example 7.27 after exposure to hydroxyapatite, and a 14% reduction of 7.30 nanoparticles after exposure to hydroxyapatite. This amount of reduction of nanoparticles in the supernatant is significant; negative control lots 7.25 and 7.26, which contain drug and UV-labels, but no targeting agents, showed on average no decrease in the amount of nanoparticles in the supernatant after exposure to hydroxyapatite.

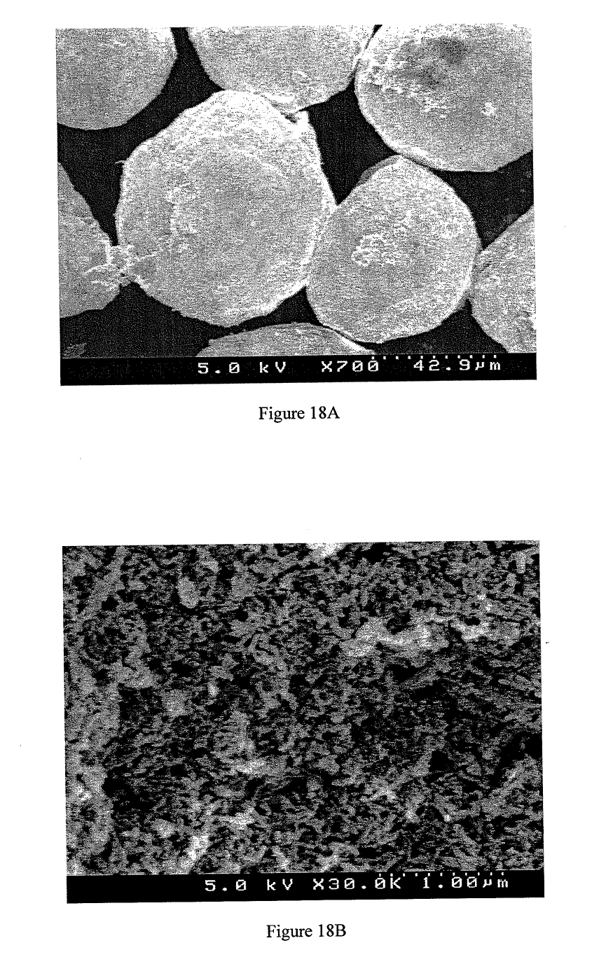

The hydroxyapatite particles resulting from these assays were isolated from the supernatant, dried under a stream of dry air, and analyzed using scanning electron microscopy (SEM.) The SEM images are shown in FIGS. 18 to 21. In each set of figures, the first figure shows the entire particle and the second figure shows the surface of the particle. Note the scale in the lower right corner of each photo.

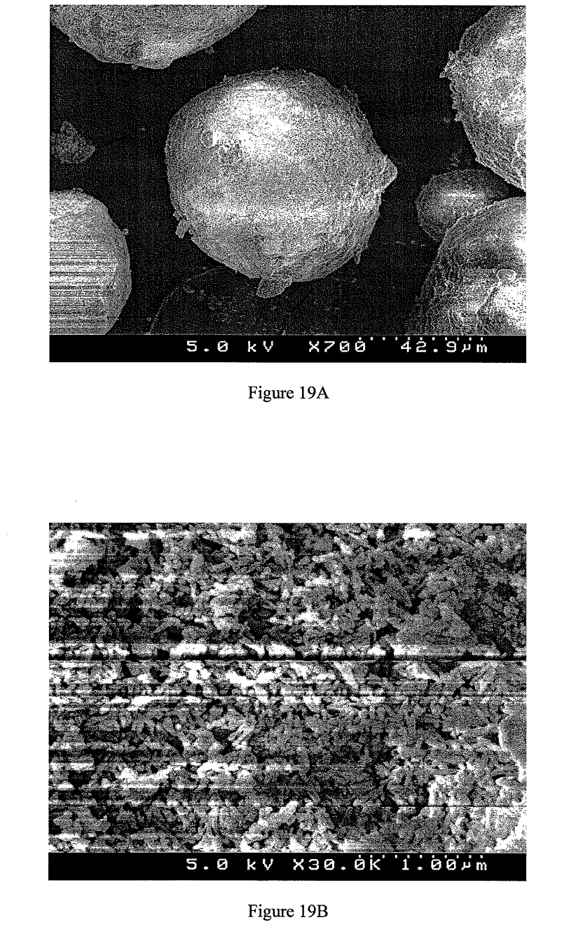

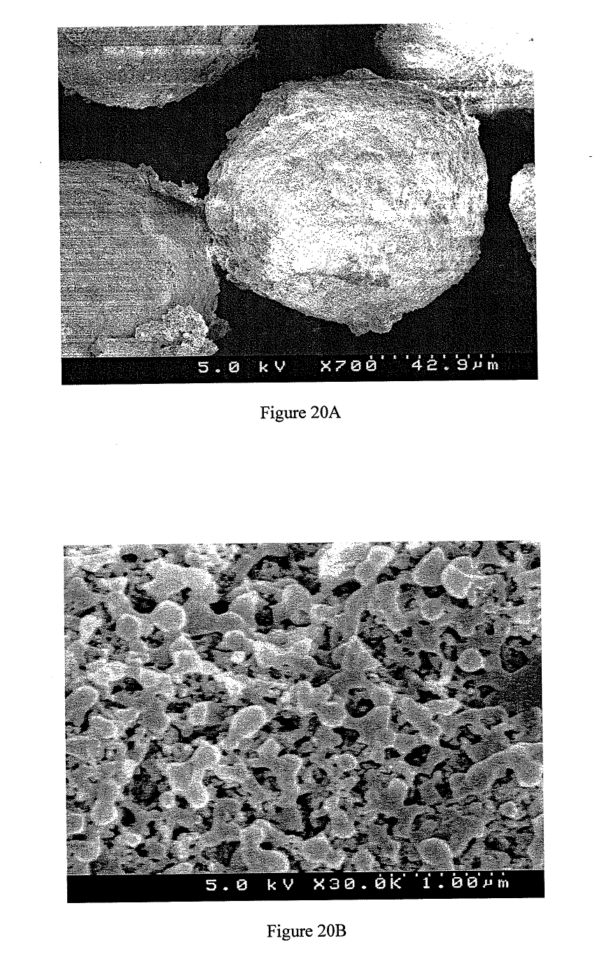

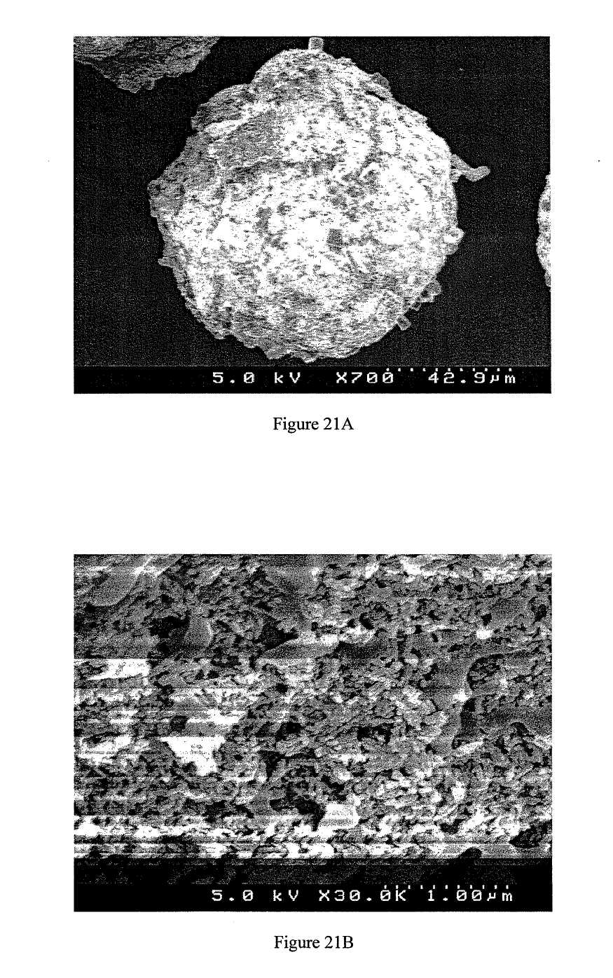

FIGS. 18A and 18B show the BioRad hydroxyapatite particles as received. FIGS. 19A and 19B shows the surface of the hydroxyapatite particles after exposure to nanoparticles prepared in Example 7.26. The nanoparticle lacked any bone targeting groups in this control, and produced a hydroxyapatite surface without nanoparticles. In comparison, FIGS. 20A and 20B after exposure to bone targeting nanoparticles in Example 7.27, and FIGS. 21A and 21B after exposure to bone targeting nanoparticles from Example 7.30, clearly demonstrated the binding of the bone targeting nanoparticles to the hydroxyapatite surface.

### EXAMPLE 10

This example illustrates a method for preparing nanoparticles containing pifithrin-α or pifithrin-β.

Biodegradable nanoparticles containing pifithrin-α and pifithrin-β are prepared by dissolving appropriate amounts of PLA, PGA, and PLGA in acetone or ethyl acetate and subsequently adding appropriate amounts of drug, which are then dissolved in the polymer/solvent solution. The release behavior of the nanoparticles is altered by changing the amount of solvent, the amount of drug, the ratio of PLA to PGA, the amount of reactive PEG, and the physical conditions during the nanoparticles preparation such as mixing speed and temperature.

Specifically, to give nanoparticle formulations with sizes under 500 nm, 100 mg of PLA is dissolved in 1.5-3.0 ml of ethyl acetate. The biodegradable polymer used is commercially available poly(lactic acid), acid end-capped, 10-20,000 molecular weight. Concurrently, 10-30 mg of mPEG-SPA, molecular weight 5,000, from Nektar, formerly Shearwater Polymers, is dissolved in 1 ml of methanol, which is added to the polymer solution and mixed. After the polymer dissolves, 5-15 mg of pifithrin-α or pifithrin-β is added to the polymer solution and allowed to stand with moderate swirling until the drug dissolves. The drug/polymer mixture is then poured into 50 ml of a 10 mg/ml aqueous bovine serum albumin or 1.0% poly(vinyl alcohol) solution and stirred for approximately 30 minutes under moderate vacuum at 500 RPM to allow extraction and evaporation of the organic solvents.

### EXAMPLE 11

This example illustrates a method for preparing nanoparticles containing PLA and a PEG-modified bone targeting agent.

To a solution of 100 mgs of PLA dissolved in 2 ml ethyl acetate is added 20 mgs of PEG-diphosphonic acid complex from Example 5 in 1 ml of methanol. The solution is then poured into 50 ml of 1.0% poly(vinyl alcohol) aqueous solution and stirred under moderate vacuum at 500 rpm for 45 minutes. The resultant nanoparticles are isolated from the aqueous solution by centrifuge and lyophilization.

### EXAMPLE 12

This example illustrates a method for preparing bone targeting nanoparticles containing 5-androstenediol.

To a solution 100 mgs of PLGA (85:15 lactic acid:glycolic acid, inherent viscosity 0.66-0.80, lauryl ester end capped, from Alkermes) in 1 ml ethyl acetate is added 25 mgs of PEG-tetraphosphonic acid complex from Example 6 and 10 mgs of 5-androstenediol in 1 ml of methanol. The solution is then poured into 50 ml of a 10 mg/ml aqueous bovine serum albumin and stirred under moderate vacuum at 500 rpm for 45 minutes. The resultant nanoparticles are isolated from the aqueous solution by centrifuge and subsequent lyophilization.

### EXAMPLE 13

This comparative example illustrates the release profiles for nanoparticles containing doxirubicin or epirubicin.

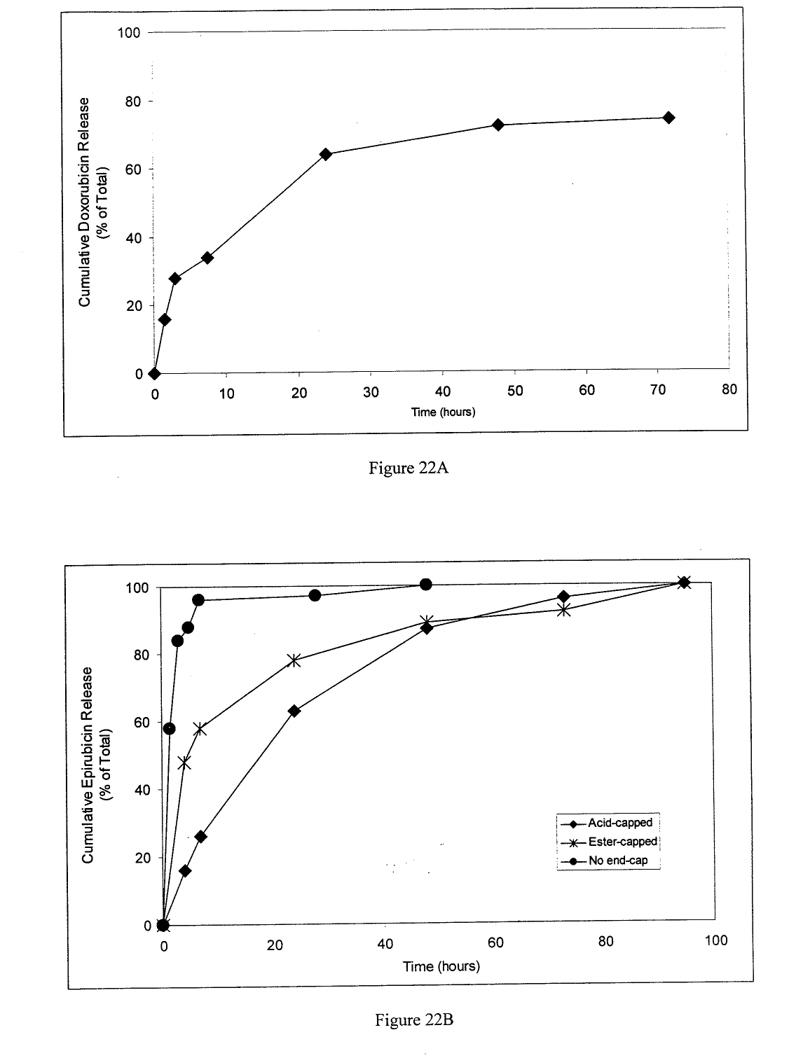

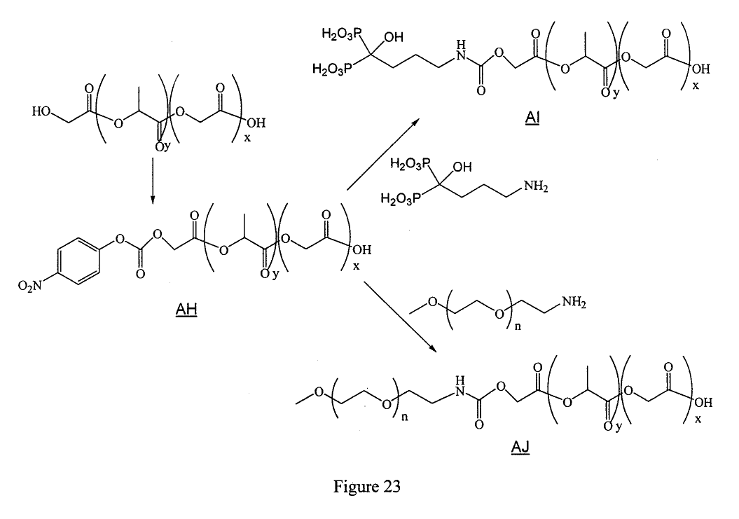

As an example of the drug release rates possible in the present invention, nanoparticles were prepared that contain doxorubicin by a variety of techniques which show desirable targeting capabilities and in vitro release profiles possible for the present invention. The release data shown in FIG. 22A is for the release in vitro of doxorubicin from nanoparticles with an average diameter of 210 nm (Brannon-Peppas et al., J. Nanopart. Res., 2, 173 (2000)). Epirubicin has also been successfully encapsulated, behaves very similarly to doxorubicin during formulation, and has both extended the release in vitro and eliminated the initial burst of release as shown in FIG. 22B.

### EXAMPLE 14

This example illustrates a method for attaching a bone targeting agent to a nanoparticle containing PEG-SPA.

An amino-containing bone targeting agent, such as those shown in FIG. 7, or FIG. 8, or U of FIG. 15 or AD or AG of FIG. 17 or ABDTMP of FIG. 3, etc., is dissolved in deionized water at a concentration of at least 1 mg/ml. After the nanoparticles in Example 10 above are prepared, the pH of the solution is adjusted carefully to 8.0. Then, 0.1-1 ml of the amino-containing bone targeting agent solution (excess relative to nanoparticle reactive groups) is added to the stirring mixture and the reaction is allowed to proceed for 1 hour. The supernatant is sampled to determine the course of the reaction progress. These techniques have been used to successfully attach fibrinogen and the Her2 antibody (Herceptin) to SSA-PEG and this method is a modification of published techniques (Hermanson, G. T., Bioconjugates, Academic Press, San Diego (1997)).

### EXAMPLE 15

This example illustrates a method for preparing an activated ester of PGLA and subsequent preparation of a bone targeting agent PLGA conjugate (AI) or PEG modified PLGA (AJ) in FIG. 23.

Polylactic-polyglycolic acid (PLGA) was obtained from Alkermes as Medisorb polymer catalog 5050DL2A (lot 9007-394) which had a molecular weight of 11 kD, polydispersity of 1.7, mole ratio of D,L-lactide of 53% and glycolide ratio of 47% with Tg of 41.3 C and inherent viscosity of 0.17 dL/g and had no endcap (i.e. the polymer has an alcohol terminus at one end and a carboxylic acid at the other end). Various other polymers with different lactide to glycolide ratio as well as various viscosities and various ester endcaps for the carboxylic acid group are commercially available from Alkermes and are suitable to substitute in place of the particular PLGA used herein. This procedure was based on H. S. Yoo et. al. in Pharmaceutical Research Vol 16(7), 1999 pp 1114-1118.

A solution of 10.0 g of PLGA dissolved in 100 mL of methylene chloride was treated with 720 mg of p-nitrophenylchloro formate (Aldrich) and cooled to 0 C in an ice bath. The solution was treated with 480 uL of pyridine in a dropwise fashion and then allowed to warm to room temperature over three hours. The reaction mixture was transferred to a separatory funnel and was then washed with 1N HCl and then with brine. The organic layer was dried over sodium sulfate and then rotoevaporated to yield 10.53 g of the PGLA-nitrophenylformate (AH) as a white solid foam. This material gave a single peak with a retention time on HPLC-MS of 5.517 minutes and the mass spectrum of this peak was indicative of a polymeric structure. (See FIG. 23.)

The PGLA-nitrophenylformate can be subsequently reacted with a bone targeting agent such as alendronate to produce a bone targeting PGLA (AI), or other amine-bearing bone targeting agents such as those shown in FIG. 7, or FIG. 8, or U of FIG. 15 or AD or AG of FIG. 17 or ABDTMP of FIG. 3, etc. The PGLA-nitrophenylformate can alternatively be reacted with mPEG-NH₂ to produce mPEG-PLGA (AJ).

### EXAMPLE 16

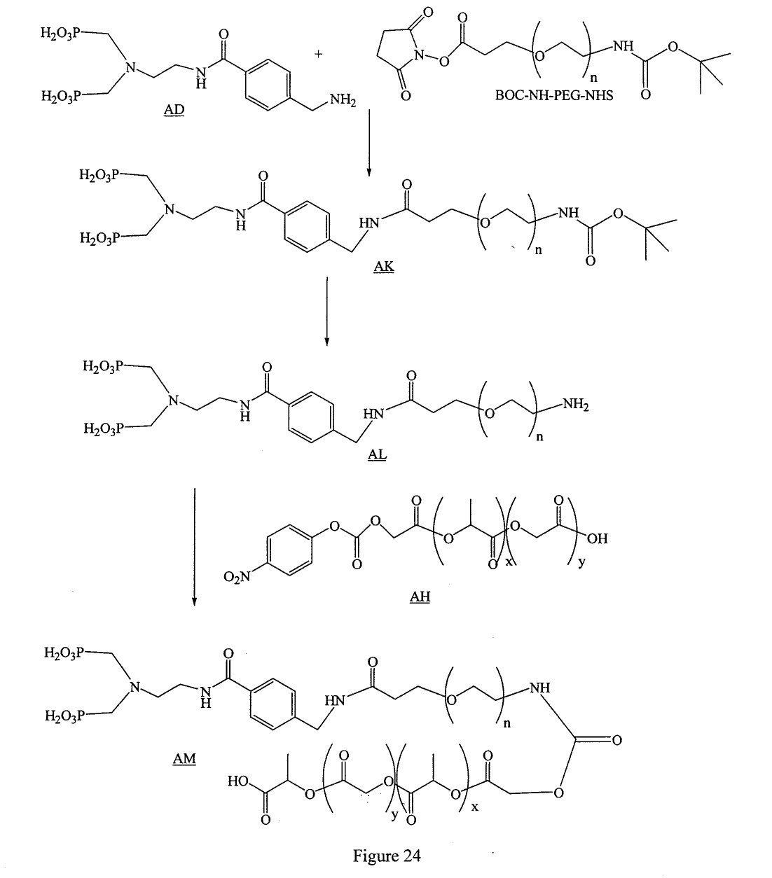

This example illustrates a method for preparing aminodiphosphonic acid-PEG-PGLA (AM) in FIG. 24.

A 20 uMole portion of the amino diphosphonic acid (AD—see FIG. 24) prepared in Example 5 dissolved in 130 μL of water was added to 250 μL of 1M sodium bicarbonate and then treated all at once with 69 mg (20 uMoles) of Boc-NH-PEG-NHS (Nektar Therapeutics, catalog number 4M530F02, 3100 kD molecular weight). The solution was allowed to stand 16 hours and then lyophilized to give 90 mg of white solid diphosphonate-PEG-NHS (AK). A small sample was dissolved in water and analyzed by HPLC-MS and the desired product was observed with a retention time of 3.175 minutes versus a retention time of 2.970 for the starting polymer and the new peak showing strong UV activity at both 254 and 214 nm indicative of the aromatic group of the bone agent being covalently attached to the PEG polymer. The mass spec data associated with this peak gave a pattern consistent with a polymeric material. All of this material was dissolved in 1.5 mL of trifluoroacetic acid and allowed to stand for 1.5 hours at room temperature. A 10 uL aliquot was removed and blown dry with argon stream and dissolved in 400 uL of water and analyzed by HPLC-MS. The analysis showed that all of the starting material was gone and replaced by a new peak at 2.553 minutes showing UV absorption consistent with removal of the Boc protecting group. The reaction mix was blown down to give 194 mg of the bone targeting-PEG-NH₂ (AL). The absence of the Boc protecting group was confirmed by proton NMR in D2O [¹H NMR: 2H δ 3.43 ppm Singlet; 4H 2.052 ppm Singlet; 2H 23.080 ppm triplet; 2H 2.468 ppm triplet; 438H, 3.568 broad singlet (polymer backbone —OCH2CH2— unit); 2H 7.67 ppm doublet, 2H 7.30 ppm doublet]. All of this sample was then dissolved in 1 mL of dry acetonitrile and treated with 220 mg of PLGA nitrophenyl formate (AH) and then treated with 3-100 μL portions of neat triethylamine. The solution was allowed to stand for 11 hours and then samples and analyzed by HPLC-MS. This analysis showed complete loss of the bone targeting PEG-NH₂ (AH) [retention time 2.553 minutes] and slight UV absorbance for the PLGA peak with a retention time (broad peak) of 5.560 minutes. Moreover, the appearance of para-nitrophenol as a result of the displacement by the PEG-NH₂ nucleophile attack to give the desired covalently bound new PLGA construct was quantitated by HPLC and corresponded to 86% of theory also indicating the desired reaction had taken place to a great extent. The reaction mix was then rotoevaporated to give 489 mg of sticky yellow solid assigned as the bone targeting PEG-PLGA (AM).

### EXAMPLE 17

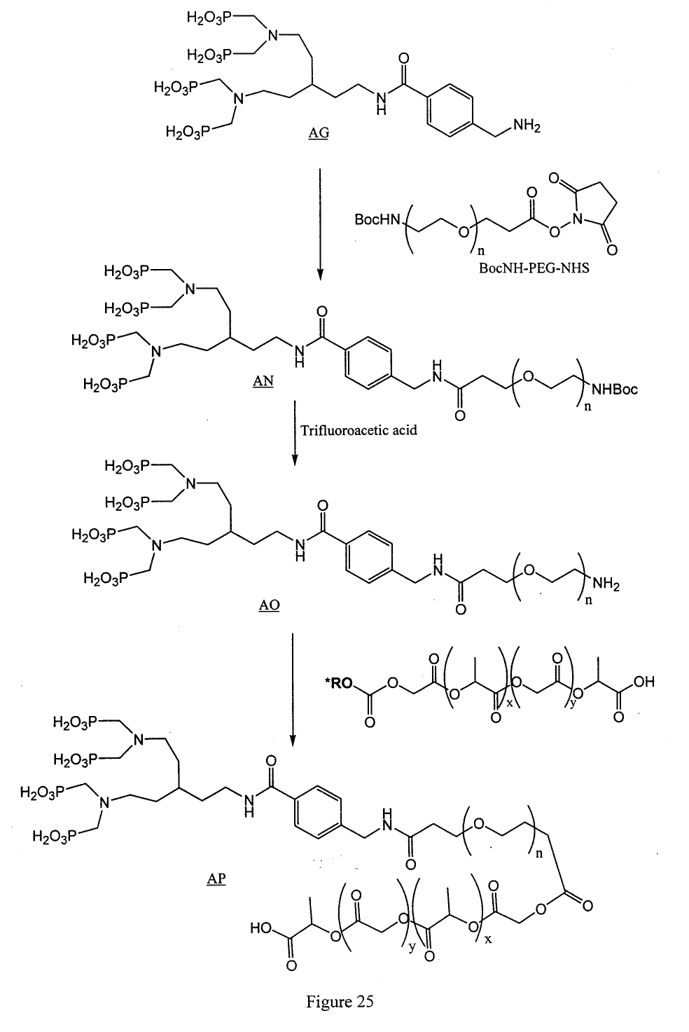

This example illustrates a method for preparing aminotetraphosphonic acid-PEG-PGLA (AP) in FIG. 25.

A 36 mg (55 uMole) portion of amine tetraphosphonic acid (AG—see FIG. 25) prepared in Example 6 was dissolved in 1 mL of 1M sodium bicarbonate to give a solution with a pH of about 8-9 by pH paper. To this was added 170 mg of Boc-NH-PEG-NHS (Nektar Therapeutics, catalog number 4M530F02, 3100 kD molecular weight) which went into solution with vigorous stirring. After 11 hours at room temperature the reaction was analyzed by HPLC-MS which showed that >60% of the starting PEG had been converted to a broad new peak (retention time 4.03 minutes) possessing UV activity at 254 nm characteristic of covalently incorporating the phenyl group of the bone targeting agent onto the polymer. The mass spectra of this new peak also indicated a polymeric structure. This solution was assigned to Boc-NH-PEG-aminotetraphosphonic acid (AN). All of this material was purified by preparative HPLC-MS to give 47.2 mg of white solid.

A 47.2 mg portion of the Boc-NH-PEG-aminotetraphosphonic acid was dissolved in 1 mL of trifluoroacetic acid and allowed to stand at room temperature for 30 minutes. An aliquot was removed and blown down with argon and dissolved in acetonitrile and analyzed by HPLC-MS. This analysis showed that all of the starting material had been converted to a new peak with retention time of 2.985 minutes showing UV activity and mass spectra consistent with a polymer. The reaction solution was rotoevaporated to give 52.7 mg of the whites solid NH₂-PEG-iminotetraphosphonic acid (AO).

A 40 mg portion of NH₂-PEG-aminotetraphosphonic acid was dissolved in 150 μL of dry acetonitrile and treated with 102 mg of PGLA-nitrophenylformate (AH) to give a clear solution. This solution was treated with 200 μL of triethylamine and the solution allowed to stand at room temperature. After 15 hours an aliquot was analyzed by HPLC-MS and showed complete disappearance of the starting material and showed slight UV absorption associated with the PLGA peak (retention time 5.356 minutes, broad). The desired reaction was further confirmed by the appearance of para-nitrophenol indicating conjugation had occurred. The reaction solution was rotoevaporated to give 147 mg of sticky yellow solid assigned PGLA-PEG-aminotetraphosphonic acid (AP).

### EXAMPLE 18

This example illustrates a preparation of bone targeting nanoparticles composed of PLA and a bone targeting PEG.

To a solution of 1 ml ethyl acetate and 1 ml methanol is added 100 mgs of PLA, 50 mgs of a PEG conjugate of the amine tetraphosphonic acid (AG) from Example 7, and 10 mgs of pifithrin β, and the solution sonicated for 90 seconds. The resulting solution is then poured into 50 ml of 1.0% poly(vinyl alcohol) aqueous solution and stirred under moderate vacuum at 500 rpm for 45 minutes. The resultant nanoparticles are isolated from the aqueous solution by centrifuge and lyophilization.

### EXAMPLE 19

This example illustrates a method for preparing bone targeting nanoparticles composed of a PEG-modified PLGA, PLGA, and a bone targeting PEG.

To a solution of 1 ml acetone is added 50 mgs of PEG-PLGA (AJ) from Example 15, 50 mgs of amino diphosphonic acid-PLGA (AI) from Example 15, and 20 mgs of amino diphosphonic acid-PEG (V) from Example 3. This mixture is sonicated for 60 seconds to completely dissolve the reagents, then 10 mgs of pifithrin β dissolved in 1 ml methanol is added. The resulting solution is then poured into 50 ml of 1.0% poly(vinyl alcohol) aqueous solution and stirred under moderate vacuum at 500 rpm for 45 minutes. The resultant nanoparticles are isolated from the aqueous solution by centrifuge and lyophilization.

### EXAMPLE 20

This example illustrates a method for preparing bone targeting nanoparticles composed of a PEG-modified PLGA, a bone targeting PLGA, and a bone targeting PEG-modified PLGA.

To a solution of 1 ml acetone and 1 ml methanol is added 50 mgs of PEG-PLGA (AJ) from Example 15, 25 mgs of amino diphosphonic acid-PLGA (AI) from Example 15, and 50 mgs of aminotetraphosphonic acid-PEG-PLGA (AP) from Example 17. This mixture is sonicated for 60 seconds to completely dissolve the reagents, then 15 mgs of pifithrin β is added and allowed to dissolve. The resulting solution is then poured into 50 ml of 1.0% poly(vinyl alcohol) aqueous solution and stirred under moderate vacuum at 500 rpm for 45 minutes. The resultant nanoparticles are isolated from the aqueous solution by centrifuge and lyophilization.

### EXAMPLE 21

This example illustrates a method for preparing bone targeting nanoparticles composed of PLGA and two different bone targeting PEG conjugates.

To a solution of 1 ml acetone and 1 ml methanol is added 100 mgs of PLGA (65/35 lactic/glycolic, methyl ester end groups), 25 mg of amino diphosphonic acid-PEG (V) from Example 3, and 25 mgs mPEG-SPA. This mixture is sonicated for 60 seconds to completely dissolve the reagents, then 20 mgs of pifithrin β is added and allowed to dissolve. The resulting solution is then poured into 50 ml of 1.0% poly(vinyl alcohol) aqueous solution containing 3 mg of amine tetraphosphonic acid (AG) from Example 6, and stirred under moderate vacuum at 500 rpm for 45 minutes. The resultant nanoparticles are isolated from the aqueous solution by centrifuge and lyophilization.

### EXAMPLE 22

This example illustrates a method for preparing bone targeting nanoparticles composed of a PEG-modified PLA, PLGA, and a bone targeting PEG.

To a solution of 1 ml acetone and 1 ml methanol is added 50 mgs of a PLA-PEG conjugate, 50 mgs of PGLA from Alkermes as Medisorb polymer catalog 5050DL2A, and 50 mgs mPEG-SPA. This mixture is sonicated for 60 seconds to completely dissolve the reagents, then 30 mgs of pifithrin β is added and allowed to dissolve. The resulting solution is then poured into 50 ml of 1.0% poly(vinyl alcohol) aqueous solution containing 6 mg of amine tetraphosphonic acid (AG) from Example 6, and stirred under moderate vacuum at 500 rpm for 45 minutes. The resultant nanoparticles are isolated from the aqueous solution by centrifuge and lyophilization.

### EXAMPLE 23

This example demonstrates methods for characterizing nanoparticles.

The nanoparticle size distribution is analyzed using a Coulter Nanosizer, which reports a median diameter and a relative polydispersity. (A polydispersity of 1 represents a monodisperse sample.) The Coulter Nanosizer is calibrated with 200 nm latex spheres (Polyscience, Warrington, Pa.) In some instances, aggregation of the sample may be observed, and can produce a median particle diameter of greater than 1 micron and a relative polydispersity of above about 9.

The specific methods by which the particles are prepared can be modified in order to maximize the percentage of particles that are smaller than 500 nm, preferably less than 300 nm, in diameter. In addition, particles are tested using hydrophobic interaction chromatography to evaluate the relative amount of PEG at the particle surface. Hydrophobic interaction chromatography (HIC) is used to detect the presence of PEG at the particle surface. Samples are prepared by dispersing particles in saline at approximately 2 mg/ml, filtering the solution with 1.2 μm glass fiber filter paper, and injecting 1 ml of this solution onto the HIC column. The opacity of subsequent saline washes through the column is measured at 400 nm on a UV-Vis spectrophotometer. Specifically, a 1 ml-capacity butyl or phenyl sepharose column is charged with the particle solution. Saline is pumped through the column at 0.8 ml/min and the effluent is collected continuously in 5 minute intervals for 10 min. Then 1 ml of Triton X (0.01% v/v in phosphate buffered saline) is used as a first wash to remove any slightly bound particles. Another 5 minute saline wash is followed by 1 ml of 0.05% Triton to remove moderately bound particles. Another 5 ml saline wash is followed by a 1 ml wash of 0.1% Triton to remove all remaining particles. A final 10 ml wash (with all washes at 0.8 ml/min) is performed to remove any particles remaining in the column. All samples and rinses are measured for opacity to determine the relative percentage of particles that interacted with the column. Since PEG is hydrophilic, particles with PEG on their surface pass through the column unaffected. Untreated particles, with a hydrophobic PLGA surface, interact and bind with the column packing and remain until a detergent (Triton X) is used to wash the particles off the column.

The amount of PEG present in the nanoparticles is assayed by a colorimetric method that takes advantage of the formation of a complex between iodine and PEG (Brannon-Peppas et al., J. Nanoparticle Res., 2, 173 (2000)).

Since the presence of PEG and organic moieties and the overall surface charge is important to the eventual uptake of these nanoparticles in vivo, the surface of these particles is analyzed using a ZetaPlus potential analyzer (Brookhaven Instruments). Nanoparticles are studied for their zeta potential to characterize their surface as prepared, after freeze-drying and resuspension.

### EXAMPLE 24

This example illustrates a method for evaluating the binding, retention, degradation, “stealthiness”, and other structure-performance relationships of the bone targeting nanoparticles.

Nanoparticles (NP) prepared by varying a limited number of variables are treated with an excess of bone targeting agents in order to cap all available PEG reactive groups. These nanoparticles bearing pendant bone targeting agents (NP-BTA) are studied to determine (quantitate) the binding and retention on hydroxyapatite surfaces. The best performing NP-BTAs that yield strong hydroxyapatite binding and retention, defined as approaching within 10-fold that of current clinically used bone targeted small molecules (EDTMP, DOTMP), are examined further. This further examination determines degradation rates of the best NP-BTAs under physiological conditions both in suspension and attached to a hydroxyapatite surface. Next the amount of BTA relative to the PEG is varied and the resulting nanoparticles evaluated for binding and degradation. All particles prepared are evaluated for their phagocytic potential (a measure of stealthiness) using a simple in vitro macrophage cell test to help understand the factors that contribute to undesirable phagocytosis of nanoparticles which in vivo could potentially compete with the selective targeting of such particles to bone and bone marrow. This experiment explores the relationship between the stealthiness of nanoparticles and having enough bone targeting agent to direct the nanoparticle to the bone surface.

### EXAMPLE 25

This example illustrates a method for measuring the degradation of the biodegradable nanoparticle.

Only nanoparticles with bone targeting agents attached that approach ten times of the bone affinity of clinically known small molecule bone targeting agents are analyzed and quantified for their ability to degrade.

Dialysis cells with 1 ml-capacity cavities (Bel-Art Products, Pequannock, N.J.) are fitted with Spectra/Por®Biotech cellulose ester dialysis membranes (Spectrum, Laguna Hills, Calif.) and used in drug release studies. Particles (20-50 mg) are suspended in 1 ml of saline and injected into one cavity (donor side). Fresh saline is injected into the other cavity (recipient). The cells are placed in a heated, shaking water bath (37° C.). At predetermined times, the recipient solution is removed and completely replaced with fresh saline. Samples are filtered through 0.45 μm syringe filters and the absorption of each is measured by HPLC. A portion of the release samples is also analyzed for the presence of mPEG-SPA, bone targeting moieties and mPEG-SPA/complexes. An HPLC size exclusion technique using Waters Ultrahydrogel columns allows the identification of the separate peaks for BSA and SSA-PEG with some overlap of the peaks. An HPLC coupled with mass spectrometry and evaporative light scattering detector allows for analysis and quantitative measurement of the bone targeting agent.

### EXAMPLE 26

This example illustrates methods for evaluating the stealthiness of the nanoparticles.

The bone targeting nanoparticles are evaluated in a biological test to evaluate their detrimental potential for macrophagic engulfment as a model for the process involved in spleen and liver uptake. Briefly, the bone targeting nanoparticles loaded with pifithrin are exposed to cell cultures of the J774 macrophage cell line. At various time points the cells are separated from the supernatant by simple filtration. The filtrate is then split and one portion analyzed for the total amount of pifithrin present. The other portion of the filtrate is ultracentrifuged to separate the pifithrin still present in nanoparticles and then analyzed for soluble pifithrin. In this manner, with the proper controls the amount of pifithrin associated with the J774 cells (assumed to be phagocytized), the amount of pifithrin in solution, and the amount of pifithrin still associated with the nanoparticles is calculated. This quantitation of phagocytic potential provides an understanding of the relationship between nanoparticle structure (including surface characteristics) and undesirable phagocytosis which is invaluable in focusing on the SBIR phase II optimization work including animal studies. This testing in concert with the ability to vary the amount of PEG coating and degree of bone affinity group modification maximizes both the “stealthiness” of the particles (as measured by amount of phagocytosis) and the bone targeting properties (degree of PEG substitution with bone affinity groups) of the nanoparticles.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of modifying a cellular response in a mammal comprising administering to the mammal an effective amount of biodegradable nanoparticles, each of said nanoparticles comprising an active agent, a biodegradable polymer, and a bone targeting agent.

2. The method of claim 1, wherein modifying a cellular response comprises temporarily inhibiting a p53 protein.

3. The method of claim 1, wherein modifying a cellular response comprises activating a p53 protein.

4. The method of claim 1, wherein modifying a cellular response comprises stimulating bone marrow cells.

5. The method of claim 4, wherein the active agent is a granulocyte stimulating factor.

6. The method of claim 1, wherein the biodegradable nanoparticle comprises PEG and a polyester selected from the group consisting of PLA, PLGA, PGA, and mixtures thereof.

7. The method of claim 7, wherein the biodegradable nanoparticle comprises PEG and PLGA.

8. The method of claim 6, wherein the biodegradable nanoparticle comprises PLGA having a lactic acid:glycolic acid molar ratio of about 95:5 to about 5:95.

9. The method of claim 8, wherein the biodegradable nanoparticle comprises PLGA having a lactic acid:glycolic acid molar ratio of about 75:25 to about 25:75.

10. The method of claim 9, wherein the biodegradable nanoparticle comprises PLGA having a lactic acid:glycolic acid molar ratio of about 50:50.

11. The method of claim 6, wherein the biodegradable nanoparticle comprises PLGA or PLA, and wherein the lactic acid component is racemic, enantiomerically enriched, or enantiopure.

12. The method of claim 6, wherein the PEG has a molecular weight of about 700 to about 100,000.

13. The method of claim 12, wherein the PEG has a molecular weight of about 1000 to about 20,000.

14. The method of claim 1, wherein the bone targeting agent is selected from the group consisting of a phosphate, a phosphonate, a bisphosphonate, a hydroxybisphosphonate, an aminomethylenephosphonate, an acidic peptide, and any combination thereof.

15. The method of claim 14, wherein the bone targeting agent is covalently bound in the nanoparticle.

16. The method of claim 14, wherein the bone targeting agent is covalently bound to the biodegradable polymer.

17. The method of claim 14, wherein the bone targeting agent is covalently bound to the surface of the polymer.

18. The method of claim 14, wherein the bone targeting agent is dispersed within the polymer matrix.

19. The method of claim 15, wherein the biodegradable nanoparticle comprises PEG and the bone targeting agent is covalently bound to at least 10% of the PEG.

20. The method of claim 19, wherein the bone targeting agent is covalently bound to at least 25% of the PEG.

21. The method of claim 20, wherein the bone targeting agent is covalently bound to at least 50% of the PEG.

22. The method of claim 2, wherein the active agent is Formula I: 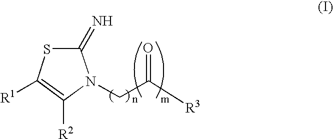
wherein m is 0 or 1, n is an integer from 1 to 4,
R¹ and R² taken together, form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and optionally forms a C₃-C₆ cycloalkyl when R³ is connected to the carbon β to the thiazole ring.

23. The method of claim 22, wherein the active agent is Formula II:

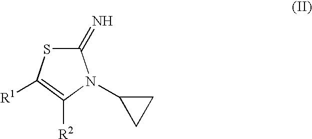

24. The method of claim 2, wherein the active agent is Formula III: 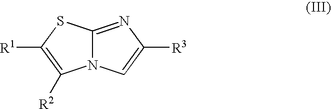
wherein R¹ and R² taken together form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties.

25. The method of claim 22, wherein R¹ and R² taken together form a 5- or 6-membered aliphatic carbocyclic ring optionally substituted with one or more C₁-C₆ alkyl groups.

26. The method of claim 22, wherein the active agent is Formula IV: 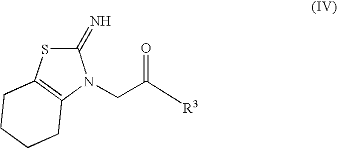
wherein R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic groups.

27. The method of claim 22, wherein the active agent is Formula V: 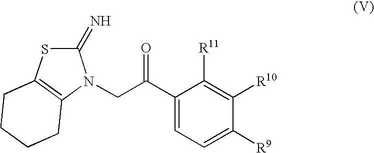
wherein R⁹, R¹⁰, and R¹¹ are each independently selected from the group consisting of a hydrogen, hydroxyl, methyl, fluoro, chloro, bromo, nitro, amino, methoxy, aryl, and heteroaryl.

28. The method of claim 27, wherein R⁹ is methyl.

29. The method of claim 27, wherein R⁹ is phenyl.

30. The method of claim 22, wherein the active agent is 2-[2-imino-4,5,6,7-tetrahydro-1,3-benzothiazol-3(2H)-yl]-1-(4-methylphenyl)-1-ethanone or 2-[2-imino-4,5,6,7-tetrahydro-1,3-benzothiazol-3(2H)-yl]-1-(biphenyl)-1-ethanone.

31. The method of claim 24, wherein the active agent is 2-p-tolyl-5,6,7,8-tetrahydro-benzo[d]imidazo[2,1-b]thiazole

32. The method of claim 22, wherein the active agent protects bone marrow cells during chemotherapy and/or radiation therapy.

33. A composition comprising an active agent, a biodegradable nanoparticle, and a bone targeting agent.

34. The composition of claim 33, wherein the biodegradable nanoparticle comprises PEG and a polyester selected from the group consisting of PGA, PLA, PLGA, and mixtures thereof.

35. The composition of claim 34, wherein the biodegradable nanoparticle comprises PEG and PLGA.

36. The composition of claim 34, wherein the biodegradable nanoparticle comprises PLGA having a lactic acid:glycolic acid molar ratio of about 95:5 to about 5:95.

37. The composition of claim 36, wherein the biodegradable nanoparticle comprises PLGA having a lactic acid:glycolic acid molar ratio of about 75:25 to about 25:75.

38. The composition of claim 37, wherein the biodegradable nanoparticle comprises PLGA having a lactic acid:glycolic acid molar ratio of about 50:50.

39. The composition of claim 34, wherein the biodegradable nanoparticle comprises PLGA or PLA, and wherein the lactic acid component is racemic, enantiomerically enriched, or enantiopure.

40. The composition of claim 34, wherein the PEG has a molecular weight of about 700 to about 100,000.

41. The composition of claim 40, wherein the PEG has a molecular weight of about 1,000 to about 20,000.

42. The composition of claim 33, wherein the bone targeting agent is selected from the group consisting of an phosphate, a phosphonate, a bisphosphonate, a hydroxybisphosphonate, an aminomethylenephosphonate, an acidic peptide, and any combination thereof.

43. The composition of claim 42, wherein the bone targeting agent is covalently bound in the nanoparticle.

44. The composition of claim 42, wherein the bone targeting agent is covalently bound to the biodegradable polymer.

45. The composition of claim 42, wherein the bone targeting agent is covalently bound to the surface of the polymer.

46. The composition of claim 42, wherein the bone targeting agent is dispersed within the polymer matrix.

47. The composition of claim 33, wherein the active agent temporarily inhibits p53 protein.

48. The composition of claim 33, wherein the active agent activates p53 protein.

49. The composition of claim 33, wherein the active agent stimulates bone marrow cells.

50. The composition of claim 49, wherein the active agent is a granulocyte stimulating factor.

51. The composition of claim 47, wherein the active agent is Formula I: 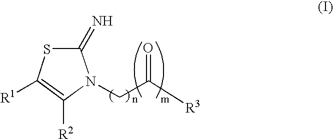
wherein m is 0 or 1, n is an integer from 1 to 4,
R¹ and R² taken together form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and optionally forms a C₃-C₆ cycloalkyl when R³ is connected to the carbon β to the thiazole ring.

52. The composition of claim 47, wherein the active agent is Formula III: 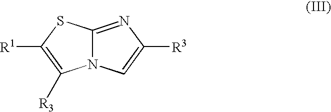
wherein R¹ and R² taken together form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties.

53. The composition of claim 33, wherein at least 95% of the nanoparticles are about 10 to about 1000 nm in diameter.

54. The composition of claim 53, wherein at least 90% of the nanoparticles are about 100 to about 400 nm in diameter.

55. A method of delivering an exogenous substance to a mammal, wherein the method comprises administering to the mammal an effective amount of a biodegradable nanoparticle comprising the exogenous substance, a biodegradable polymer, and a bone targeting agent.

56. The method of claim 55, wherein the exogenous substance comprises at least one drug, at least one protein, at least one nucleic acid, or a mixture thereof.

57. The method of claim 55, wherein the biodegradable nanoparticle comprises PEG and a polyester selected from the group consisting of PLA, PLGA, PGA, and mixtures thereof.

58. The method of claim 57, wherein the biodegradable nanoparticle comprises PLGA having a lactic acid:glycolic acid molar ratio of about 95:5 to about 5:95.

59. The method of claim 58, wherein the biodegradable nanoparticle comprises PLGA having a lactic acid:glycolic acid molar ratio of about 75:25 to about 25:75.

60. The method of claim 59, wherein the biodegradable nanoparticle comprises PLGA having a lactic acid:glycolic acid molar ratio of about 50:50.

61. The method of claim 57, wherein the biodegradable nanoparticle comprises PLGA or PLA, and wherein the lactic acid component is racemic, enantiomerically enriched, or enantiopure.

62. The method of claim 57, wherein the PEG has a molecular weight of about 700 to about 100,000.

63. The method of claim 62, wherein the PEG has a molecular weight of about 1000 to about 10,000.

64. The method of claim 55, wherein the bone targeting agent is selected from the group consisting of a phosphate, a phosphonate, a bisphosphonate, a hydroxybisphosphonate, an aminomethylenephosphonate, an acidic peptide, and any combination thereof.

65. The method of claim 64, wherein the biodegradable nanoparticle comprises PEG and the bone targeting agent is covalently bound to at least 10% of the PEG of the biodegradable nanoparticle.

66. The method of claim 65, wherein the bone targeting agent is covalently bound to at least 25% of the PEG of the biodegradable nanoparticle.

67. The method of claim 66, wherein the bone targeting agent is covalently bound to at least 50% of the PEG of the biodegradable nanoparticle.

68. A process for preparing a biodegradable nanoparticle comprising an active agent, a biodegradable polymer, and a bone targeting agent, the process comprising
(1) providing an organic phase comprising one or more of (a)-(i)
(a) a biodegradable polymer,
(b) a PEG,
(c) an activated PEG of Formula VI: 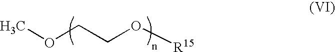
wherein n is an integer from 2 to 2000, and R¹⁵ is an organic radical that contains an electrophilically activated leaving group,
(d) a bone targeting agent,
(e) a PEG-modified biodegradable polymer,
(f) a bone targeting agent-biodegradable polymer conjugate,
(g) a bone targeting agent-PEG conjugate,
(h) a bone targeting agent-PEG-modified biodegradable polymer conjugate, and
(i) an active agent
dissolved in one or more organic solvents, with the requirement that the organic phase contains at least one PEG, at least one biodegradable polymer, and at least one active agent,
(2) mixing the organic phase of (1) with an aqueous phase comprising water and a surface active agent to form a mixture
(3) removing the organic solvent(s) from the mixture while stirring and recovering the resultant nanoparticles, and
(4) optionally treating the nanoparticles with a bone targeting agent.

69. The process according to claim 68 wherein the biodegradable polymer comprises a polyester.

70. The process according to claim 69, wherein the polyester is selected from a group consisting of PGA, PLA, PLGA, and mixtures thereof.

71. The process according to claim 68, wherein the organic phase is organic solution.

72. The process according to claim 68, wherein the aqueous phase is an aqueous solution.

73. The process according to claim 68, wherein the organic solvent is selected from the group consisting of C₁-C₄ alcohols, C₂-C₆ esters, C₂-C₆ ethers, and C₁-C₆ organic acids.

74. The process according to claim 68 wherein the surface active agent is bovine serum albumin, human serum albumin, or polyvinyl alcohol.

75. The process according to claim 74, wherein bovine serum albumin or human serum albumin is present in the aqueous phase at a concentration of about 5-15 mg/ml.

76. The process according to claim 74, wherein the polyvinyl alcohol is present in the aqueous phase at a concentration of about 0.5 to 2.0% by volume.

77. The process according to claim 68, wherein the organic phase comprises a biodegradable polymer and an activated PEG.

78. The process according to claim 77, wherein the organic phase further comprises a bone targeting agent.

79. The process according to any of claims 68, wherein the organic phase comprises a PEG-modified biodegradable polymer, a bone targeting agent-biodegradable polymer conjugate, and a bone targeting agent-PEG conjugate.

80. The process according to claim 68, wherein the organic phase comprises a PEG-modified biodegradable polymer, a bone targeting agent-PEG conjugate, and a bone targeting agent PEG-modified biodegradable polymer conjugate.

81. The process according to claim 68, wherein if an activated PEG of Formula VI is present, then R¹⁵ is succinimidyl propionate or succinimidyl butanoate.

82. The process according to claim 68, wherein the bone targeting agent is selected from the group consisting of a phosphate, a phosphonate, a bisphosphonate, a hydroxybisphosphonate, an aminomethylenephosphonate, an acidic peptide, and any combination thereof.

83. The process according to claim 68, wherein the active agent comprises at least one drug, at least one protein, at least one nucleic acid, or a mixture thereof.

84. A method of modifying a cellular response in a mammalian cell comprising contacting the mammalian cell with a biodegradable nanoparticle, said biodegradable nanoparticle comprising an active agent, a biodegradable polymer, and a cell targeting agent.

85. The method of claim 84, wherein the cell-targeting agent is a bone targeting agent.

86. The method of claim 85, wherein the contact is in vitro.

87. The method of claim 85, wherein the contact is in vivo.

88. The method of claim 24, wherein the active agent protects bone marrow cells during chemotherapy and/or radiation therapy.
